(19) United States
(12) Patent Application Publication
(10) Pub. No.: US 2012/0328631 A1
(43) Pub. Date: Dec. 27, 2012

(54) EP1 INHIBITION

(75) Inventors: Regis O'Keefe, Rochester, NY (US); Minjie Zhang, Rochester, NY (US); Edward M. Schwarz, Rochester, NY (US)

(73) Assignee: UNIVERSITY OF ROCHESTER, Rochester, NY (US)

(21) Appl. No.: 13/582,927

(22) PCT Filed: Mar. 4, 2011
(86) PCT No.: PCT/US2011/027226

§ 371 Date: Sep. 5, 2012

Related U.S. Application Data
(60) Provisional application No. 61/310,864, filed on Mar. 5, 2010.

(51) Int. Cl. A61K 39/395; C12Q 1/02; A61P 19/10; A61K 31/713; A61P 19/00; A61K 38/02; A61K 31/7088

(52) U.S. Cl. 424/172.1; 514/16.7; 435/29; 514/44 A; 514/16.9

(57) ABSTRACT

Provided herein are methods of accelerating bone fracture healing in a subject. Also provided herein are methods of treating or preventing osteoporosis in a subject. Further provided are methods of screening for an agent that accelerates bone fracture healing or treats or prevents osteoporosis.

### CROSS REFERENCE TO RELATED APPLICATIONS

This application claims the benefit of U.S. Provisional Application No. 61/310,864, filed on Mar. 5, 2010, which is incorporated by reference herein in its entirety.

### STATEMENT REGARDING FEDERALLY FUNDED RESEARCH

This invention was made with government funding under Grant No. AR048681 from the National Institutes of Health. The government has certain rights in this invention.

### BACKGROUND

Osteoporosis is a disease of the bone that leads to an increase risk of fracture. In osteoporosis the bone mineral density (BMD) is reduced, bone microarchitecture is disrupted, and the amount and variety of proteins in the bone is altered. Osteoporosis is most common in women, but may also develop in men, and may occur in anyone in the presence of particular hormonal disorders and other chronic disease or as a result of medications. Given its influence in the risk of fragility fracture, osteoporosis may significantly affect life expectancy and quality of life.

Approximately 7.9 million skeletal fractures occur each year in the United States. Nearly 10-20% of all fractures have impaired healing, including delayed union or non-union. Impaired fracture healing, which requires prolonged or repeated treatments, has a marked impact on both the quality of life and the total cost of care. Since the risk of impaired fracture healing is greatest in the elderly and those with other conditions, including osteoporosis, the reduced mobility that occurs with fractures further complicates the management of these other conditions, resulting in additional personal and societal costs.

### SUMMARY

Provided are methods for accelerating bone fracture healing in a subject. The methods comprise identifying a subject with a bone fracture and administering to the subject an agent that inhibits the level of expression or activity of an EP1 receptor. Inhibition of the level of expression or activity of the EP1 receptor as compared to a control indicates the agent accelerates the bone fracture healing in the subject.

Also provided are methods for treating or preventing osteoporosis in a subject. The methods comprise identifying a subject with or at risk for developing osteoporosis and administering to the subject an agent that inhibits the level of expression or activity of an EP1 receptor. Inhibition of the level of expression or activity of the EP1 receptor indicates the agent treats or prevents osteoporosis in the subject.

Also provided are methods of screening for an agent that accelerates bone fracture healing or treats or prevents osteoporosis. The methods comprise providing a cell of osteoblast lineage comprising an EP1 receptor; contacting the cell with an agent to be screened and determining a level of expression or an activity of the EP1 receptor in the cell. A decrease in to the level of expression or activity of the EP1 receptor as compared to a control indicates the agent accelerates bone fracture healing or treats or prevents osteoporosis.

Also provided are methods of screening for an agent that accelerates bone fracture healing in a subject. The methods comprise administering to the subject having a bone fracture an agent to be screened and determining whether the agent inhibits a level of expression or activity of an EP1 receptor at the site of the bone fracture. A decrease in the level of expression or activity of the EP1 receptor as compared to a control indicates the agent accelerates bone fracture healing.

Further provided are methods of screening for an agent that treats or prevents osteoporosis in a subject. The methods comprise administering to the subject having or at risk of developing osteoporosis an agent to be screened and determining whether the agent inhibits a level of expression or activity of an EP1 receptor. A decrease in the level of expression or activity of the EP1 receptor as compared to a control indicates the agent treats or prevents osteoporosis.

Also provided are methods of promoting stem cell differentiation along an osteoblastic lineage. The methods comprise contacting osteoblastic stem cell precursors with an agent that inhibits a level of expression or activity of an EP1 receptor. A decrease in the level of expression or activity of the EP1 receptor promotes stem cell differentiation into bone forming osteoblastic cells.

### BRIEF DESCRIPTION OF DRAWINGS

FIG. 1 shows EP1^{-/-} fractures exhibit accelerated mineralization. Femur fractures were created in 10 week old EP1^{-/-} mice and wild-type (WT) controls. Fractured femurs were harvested at 7, 14, and 21 days post-fracture. FIG. 1A shows representative radiographs of fractured femurs that demonstrate the increased mineralized callus in EP1^{-/-} mice at day 14 compared to the soft callus in wild-type fractures (arrows). Accelerated remodeling at day 21 in EP1−/− fractures is evident from the contracted callus, versus the broad callus that remains in wild-type fractures (arrows). FIG. 1B shows representative histology (40× original magnification) of fractured femurs stained with alcian blue hematoxylin/orange G eosin that demonstrate cartilage and bone formation. FIG. 1C shows graphs of histomorphometric measurements (total callus area, cartilage area, woven bone area) made from 12 sections for each group (N=4 mice/group). The results are shown as mean±SEM. Statistical significance was assessed by 2-way ANOVA followed by Dunnett's test, “a” indicates p<0.05 compared to wild-type.

FIG. 2 shows EP1^{-/-} healed fractures have different bone properties. FIG. 2A shows images of femurs harvested from EP and wild-type control mice (N=5 mice/group) at days 7, 14 and 21 post-fracture were scanned at 10.5 micron isotropic resolution using a Scanco VivaCT 40 (Scanco Medical AG). FIG. 2B shows a reconstruction of μ-CT data collected from the fracture callus region of the femur as previously described. The fracture callus bone volume and mineral density were determined. Data are presented as mean±SD. Statistical comparisons were performed using 2-way ANOVA with Bonferroni post-hoc multiple comparisons. “a” indicates significant differences compared to wild-type (p<0.05).

FIG. 3 shows EP1^{-/-} healed fractures are stronger compared to wild-type. Femurs were dissected at day 28 post-fracture from EP1^{-/-} and wild-type mice (N=5 mice/group). The torque data were plotted against the rotational deformation (normalized by the gauge length and expressed as rad/mm) to determine the (FIG. 2A) ultimate torque, (FIG. 2B) torsional rigidity (slope of the linear region of the torque-normalized rotation curve), (FIG. 2C) ultimate rotation, and (FIG. 2D) energy to failure (area under the torque-normalized rotation curve). Statistical comparisons were performed using unpaired Student's t-test with Welch's corrections (does not assume equality of variances) after verifying normality of the data using the Kolmogorov-Sminrof test. “a” indicates significant differences compared to wild-type (p<0.05).

FIG. 4 shows the expression of genes involved in chondrogenesis and osteogenesis is altered in EP1^{-/-} mice. Total RNA was extracted from EP1^{-/-} mice and wild-type controls (N=4) at various time points after fracture. Real time RT-PCR was performed as described in the methods and normalized to β-actin expression. The following primer sets were used: col2a1 (FIG. 4A), col10a1 (FIG. 4B), Runx2 (FIG. 4C), osterix (FIG. 4D), ALP (FIG. 4E), col1a1 (FIG. 4F), and osteocalcin (FIG. 4G). Statistical comparisons at each time point were performed using 2-way ANOVA followed by Dunnett's test, “a” indicates p<0.05.

FIG. 5 shows EP1^{-/-} bone marrow cultures have accelerated osteoblast differentiation. Bone marrow cells were isolated from 10-week-old EP1^{-/-} mice and control C57BL/6J mice. FIG. 5A shows the experimental design for in vitro osteoblast experiments. Cells were cultured in 2 ml α-MEM containing 10% FCS at 5×10⁶ cells/well in 6-well plates. After 7 days the media was replaced with media containing β-glycerophosphate and ascorbic acid to induce osteoblast differentiation. Cells were harvested on days 10, 12, 14, 17 and 21 after plating for alkaline phosphatase (FIG. 5B) and alizarin red staining (FIG. 5C). FIG. 5D shows the cell proliferation rate as examined by BrdU activity and CellTiter blue activity at day 10. Statistical comparisons were performed using ANOVA. Significance was denoted by the symbol “a”, p<0.05.

FIG. 6 shows EP1^{-/-} bone marrow cells have enhanced expression of osteoblast genes in culture. Bone marrow cells were isolated and cultured as described for FIG. 6. Total RNA was harvested after 10, 12, 14, 17, and 21 days in culture and real time RT-PCR was performed using the primers listed in Table 1. The following primer sets were used: ALP (FIG. 6A), col1a1 (FIG. 6B), osteocalcin (FIG. 6C), Runx2 (FIG. 6D), osterix (FIG. 6E), RANKL (FIG. 6F), and OPG (FIG. 6G). Statistical comparisons were performed using ANOVA at each time point. The symbol “a” indicates p<0.05.

FIG. 7 shows EP1^{-/-} fractures have accelerated bone remodeling. FIG. 7A shows TRAcP staining was performed on sections of fractured femurs and representative photomicrographs are shown at 200× original magnification. FIG. 7B shows a graph of osteoclast numbers counted in 12 sections per group (N=4 mice/group). The mean±SEM is shown. FIGS. 7C and 7D shows graphs of RANKL (7C) and OPG (7D) RNA levels in the fracture callus examined by real time PCR. Statistical comparisons were performed using two-way ANOVA followed by Dunnett's test and “a” indicates p<0.05.

FIG. 8 shows increased osteoclastogenesis in EP1^{-/-} mice is not a cell autonomous process. Splenocytes isolated from 2-month-old EP1^{-/-} mice and control C57BL/6J mice. Cells were cultured at 50,000 cells/well in α-MEM containing 10% FCS and 10 ng/ml MCSF in 96-well plates. 50 ng/ml RANKL was added to induce osteoclastogenesis. Five days after culture, TRAcP staining was performed (FIGS. 8A and 8B), representative photographs are shown at 100× original magnification. FIG. 8C shows a graph demonstrating similar numbers of osteoclasts were observed in EP1^{-/-} and wild-type cultures. The data in FIG. 8C represent the mean of 4 different experiments (8 culture wells/group/experiment).

FIG. 9 shows EP2 and EP4 signaling are not altered in EP1^{-/-} cells. Bone marrow cells were isolated from 10-week-old EP1^{-/-} mice and wild-type mice. After culturing for 7 days, cells were treated with PGE2 (3 mM) or vehicle for 24 hours. FIG. 9A shows a Western blot of total protein extracted from the cultured cells performed using specific antibodies against EP1, EP2 and EP4 receptors. FIG. 9B shows a graph of a cAMP-Glo Assay performed on the bone marrow cells from 10-week-old EP1^{-/-} and wild-type mice. FIG. 9C shows representative images of immunohistochemistry performed using specific antibodies against EP4 receptors on the femur fracture tissue samples collected from 10-week-old EP1^{-/-} and wild-type mice at 7, 14, and 21 days post-fracture. The representative photomicrographs are shown at 200× original magnification. Statistical comparisons were performed using Student's T-test. “a” indicates p<0.05 compared to wild-type, “b” indicates p<0.05 compared to EP1^{-/-}. β-actin served as the loading control.

FIG. 10 shows that PGE2 regulates fibronectin expression through the EP1 receptor. Bone marrow cells were collected from 10-week-old EP1^{-/-} and wild-type mice and treated with 3 μM PGE2 for 24 hours before harvest. Total protein was extracted from the cultured cells at day 17 and RNA was extracted at days 10, 12, 14, 17 and 21 after culture. Western blotting (FIG. 10A) and real-time PCR (FIG. 10B) were performed to examine the expression levels of fibronectin. Immuno-staining was performed on the bone marrow cell cultures and showed that wild-type cells expressed more fibronectin than EP1^{-/-} cells (FIG. 10C). Statistical comparisons were performed using 2-way ANOVA followed by Dunnett's test. a=P<0.05 vs. wild-type, b=P<0.05 vs. control, c=P<0.05 vs. PGE2.

FIG. 11 shows that knock down of EP1 inhibits PGE2 induced fibronectin expression. Relative RNA levels of EP1 and fibronectin were determined by real-time PCR (FIGS. 11A and 11B). Immunohistochemistry was performed using mouse fibronectin primary antibody on the femur fracture tissue samples collected from 10-week-old EP1^{-/-} and wild-type mice at 3 and 7 days post-fracture (FIG. 11C). Statistical comparisons were performed using 2-way ANOVA followed by Student's t-test. a=P<0.05 vs. wild-type, b=P<0.05 vs. control, c=P<0.05 vs. PGE2.

FIG. 12 shows that EP1 is a negative regulator of bone formation. An EP1 expression vector was transfected into the EP1^{-/-} bone marrow cells by electroporation. RNA was extracted from the bone marrow cells and showed significantly increased EP1 and fibronectin expression levels in the cells expressing exogenous EP1 (FIG. 12A). ALP staining showed fewer colonies formed in the cells expressing exogenous EP1 (FIG. 12B). Bone marrow progenitor cells at day 12 after culture were treated with SC19220 (10 μm) for 30 minutes followed by treatment with PGE2 (3 μm). After 24 hours, cells were stained with Alizarin red (FIGS. 12C and 12D). RNA extracted from the cultures was examined for osteocalcin expression (FIG. 12E). Statistical comparisons were performed using Student's t-test. a=P<0.05 vs. control, b=P<0.05 vs. PGE2.

FIG. 13 shows that fibronectin inhibits osteoblast differentiation. Increasing concentrations of fibronectin were added to bone marrow cell cultures at day 12. 24 hours later, cells were stained with Alizarin Red (FIGS. 13A and 138) or harvested for real-time PCR (FIG. 13B). Statistical comparisons were performed using Student's t-test, a=P<0.05 vs. control.

FIG. 14 shows that EP1^{-/-} mice have increased bone mineral density in both cortical and trabecular bone. Femurs of EP1^{-/-} mice and age-matched wild-type mice were analyzed for cortical and trabecular bone properties using quantitative μ-CT. Reconstruction of μ-CT data on the mid-diaphyseal femur showed significantly increased polar moment of inertia (6MO and 1YO), bone mineral density, cortical thickness and cortical area (1YO) in EP1^{-/-} compared with wild-type mice (FIG. 14A). Reconstruction of μ-CT data on the metaphyseal region of femur (FIG. 14B) showed increased bone volume/total volume (6MO and 1YO) and delayed trabecular bone loss in EP1^{-/-} mice (FIG. 14C). Consistently, reconstruction of μ-CT data on the L-4 vertebral body (FIG. 14E) showed increased bone volume/total volume in EP1^{-/-} mice (1YO) (FIG. 14F). Other trabecular parameters were also different between EP1^{-/-} and wild-type mice (FIGS. 14D and 4G). Statistical analysis was performed using 2-way ANOVA followed by Dunnett's test. 5 mice were tested in each group (N=5). a=P<0.05 V.S. wild-type, b=P<0.05 vs. 2MO.

FIG. 15 shows that EP1^{-/-} mice have altered bone biomechanical properties. 3-point bending tests showed significantly increased maximum load (2MO, 6MO and 1YO) and yield load (1YO) in EP1^{-/-} femurs (FIG. 15A). Compression tests performed on the same vertebral body as in the μ-CT analysis showed increased maximum load and maximum stiffness in EP1^{-/-} mice (FIG. 15B). Statistical analysis was performed using 2-way ANOVA followed by Dunnett's test. 5 mice were tested in each group (N=5). a=P<0.05 V.S. wild-type, b=P<0.05 vs. 2MO.

FIG. 16 shows that EP1^{-/-} mice have increased bone formation and normal bone resorption. Calcien labeling was performed on 10 week-old EP1^{-/-} and wild-type mice. Sections of calvarium and tibia from wild-type and EP1^{-/-} mice were examined by fluorescence microscopy. Mineral apposition and bone formation rates were quantified (FIG. 16A). The femurs collected from 2 month-old EP1^{-/-} mice and wild-type control mice were stained using TRAcP (FIG. 16B). Resorption surface was quantified as number of osteoclasts per bone perimeter (FIG. 16C). Statistical comparisons were performed using Student's t-test. 5 mice, 3 levels per mouse, were tested in each group, N=5. a=P<0.05 vs. wild-type. Not significant is denoted as “NS.”

FIG. 17 shows that loss of EP1 protects against ovariectomy induced bone loss. Sham or ovariectomy surgeries were performed on 4-month-old EP1^{-/-} and wild-type mice. Two months after surgery, the mice were sacrificed and their blood samples were examined for 17β-estradiol levels (FIG. 17A). Cortical bone properties, such as cortical thickness and bone mineral density were examined by μ-CT on the mid-diaphyseal region (FIG. 17B). Trabecular bone regions in the metaphyseal femur (FIG. 17C) and the vertebral body (FIG. 17F) were also examined, and the corresponding bone volume/total volume (FIGS. 17D and 17G) as well as other parameters (FIGS. 17E and 17H) were determined. Statistical analysis was performed using 2-way ANOVA followed by Dunnett's test, N=5. a=P<0.05 vs. sham, b=P<0.05 vs. wild-type.

FIG. 18 shows that EP1^{-/-} mice are resistant to ovariectomy induced decreases in bone biomechanical properties. Maximum load, yield load, energy to maximum and stiffness of cortical bone were determined by 3-point bending mechanical tests (FIG. 18A). Maximum load, yield load, energy to maximum and stiffness of trabecular bone were examined by compression tests on the vertebral bodies (FIG. 18B). Statistical analysis was performed using 2-way ANOVA followed by Dunnett's test, N=5. a=P<0.05 vs. sham, b=P<0.05 vs. wild-type.

FIG. 19 shows that ovariectomy does not affect the bone formation rate in EP1^{-/-} mice. Two months after OVX or sham surgery, EP1^{-/-} and wild-type mice were sacrificed. Calcein double labeling injections were performed at 14 and 7 days prior to this end point. Tissue sections of both calvarium and tibia were analyzed (FIG. 19A). Femur and tibia were harvested for TRAcP staining (FIG. 19B). Osteoclast numbers and resorption surface were quantified (FIG. 19C). Statistical analysis was performed using 2-way ANOVA followed by Dunnett's test, N=5 mice per group were examined. a=P<0.05 vs. sham, b=P<0.05 vs. wild-type.

### DETAILED DESCRIPTION

Provided herein are methods of accelerating bone fracture healing in a subject. The methods comprise identifying a subject with a bone fracture and administering to the subject an agent that inhibits the level of expression or activity of an EP1 receptor. Inhibition of the level of expression or activity of the EP1 receptor accelerates the bone fracture healing in the subject. Such methods are also useful where orthopedic surgery has disrupted normal bone integrity including, for example, with fixation of fractures or with joint replacement.

Also provided herein are methods of treating or preventing osteoporosis in a subject. Such osteoporosis can be related to estrogen deficiency, aging, medication induced osteoporosis, or the like. The methods comprise identifying a subject with or at risk of developing osteoporosis and administering to the subject an agent that inhibits the level of expression or activity of an EP1 receptor. Inhibition of the level of expression or activity of the EP1 receptor treats or prevents osteoporosis.

Also provided are methods of promoting stem cell differentiation along an osteoblastic lineage. The methods comprise contacting osteoblastic stem cell precursors with an agent that inhibits a level of expression or activity of an EP1 receptor. Inhibition of the level of expression or activity of the EP1 receptor promotes stem cell differentiation into bone forming osteoblastic cells. Osteoblastic stem cell precursors can, for example, include mesenchymal progenitor cells.

Optionally, the agent inhibits the level of expression of the EP1 receptor. The agent can, for example, be selected from the group consisting of a small molecule, a polypeptide, a peptidomimetic, or a combination thereof. Optionally, the agent is a nucleic acid molecule. The nucleic acid molecule can, for example, be selected from the group consisting of a short interfering RNA (siRNA) molecule, a microRNA (miRNA) molecule, or an antisense molecule. Examples of siRNA sequences include 5′-AGCUUGUCGGUAUCAUGGUTT-3′ (SEQ ID NO:21) and 5′-ACUUCUAAGCACACCAGAT7-3′ (SEQ ID NO:22).

Optionally, the agent inhibits the activity of the EP1 receptor. The agent can, for example, be selected from the group consisting of a small molecule, a polypeptide, a peptidomimetic, or a combination thereof. Examples of small molecule inhibitors include by way of example SC-51089 and SC-19220 (Caymen Chemical, Ann Arbor, Mich.), 8-chloro-2-[1-oxo-3-(4-pyridinyl)propyl]hydrazide-dibenz[b,f][1,4]oxazeprine-10(11H)-carboxylic acid, monohydrochloride and 8-chloro-dibenz[b,f][1,4]oxazepine-10(11H)-carboxy-(2-acetyl)hydrazide, respectively. The polypeptide can, for example, be an antibody or an inhibitory fragment or derivative thereof. The agent can, for example, directly inhibit receptor activity by binding the receptor without activating the receptor to block binding of an agonist. Alternatively, the agent can indirectly inhibit receptor activity by binding the EP1 receptor agonist to prevent binding of the bound molecule to the receptor. Further, the receptor activity can be inhibited by interrupting a downstream member of the receptor activation pathway.

Optionally, the methods further comprise administering to the subject a second agent to that regulates osteoblast or osteoclast differentiation or function. The regulation of osteoblast or osteoclast differentiation or function can, for example, be determined by the level of one or more markers of osteoblast or osteoclast differentiation or function. By way of example, a marker of osteoblast differentiation or function can be selected from the group consisting of alkaline phosphatase, runx2, osterix, osteocalcin, bone sialoprotein, and type 1 collagen. Representative markers of osteoclast differentiation or function can be selected from the group consisting of tartrate resistant acid phosphates (TRAP), cathepsin K, and calcitonin receptor.

Also provided are methods of screening for an agent that accelerates bone fracture healing or treats or prevents osteoporosis. The methods comprise providing a cell of osteoblast lineage comprising an EP1 receptor; contacting the cell with an agent to be screened; and determining a level of expression or activity of the EP1 receptor in the cell. A decrease in the level of expression or activity of the EP1 receptor as compared to a control indicates the agent accelerates bone fracture healing or treats or prevents osteoporosis. The agent can, for example, be selected from the group consisting of a small molecule, a polypeptide, a peptidomimetic, or a combination thereof. Optionally, the level of expression of RNA encoding the EP1 receptor is determined. Optionally, the level of expression of the EP1 receptor protein is determined. Optionally, the level of activity of the EP1 receptor is determined. Further provided are in vivo screening methods, wherein the agent to be screened is administered to a subject with a fracture or with or at risk of developing osteoporosis. Such in vivo screening methods comprise detecting fracture healing or improvement in bone density and can be used in conjunction with in vitro screening methods.

Further provided are in vivo methods of screening for an agent that accelerates bone fracture healing. The methods comprise administering to a subject an agent to be screened, wherein the subject has a bone fracture; and determining whether the agent inhibits the level of expression or activity of an EP1 receptor at the site of the bone fracture. A decrease in the level of expression or activity of the EP1 receptor as compared to a control indicates the agent accelerates bone fracture healing. The agent can, for example, be selected from the group consisting of a small molecule, a polypeptide, a peptidomimetic, or a combination thereof. Optionally, the level of expression of RNA encoding the EP1 receptor is determined. Optionally, the level of expression of the EP1 receptor protein is determined. Optionally, the level of activity of the EP1 receptor is determined.

In in vivo screening methods, the methods optionally further comprise detecting in the subject a formation of a hard callus at the site of the bone fracture. Optionally, the methods to further comprise detecting a level of mineralization or bone remodeling at the site of the fracture. Mineralization can, for example, be detected by determining an increase in total cartilage area. An increase in total cartilage area as compared to a control indicating an increase in mineralization. Bone remodeling can, for example, be detected by determining an increase in osteoclast cell numbers. An increase in osteoclast cell numbers as compared to a control indicates an increase in bone remodeling.

Further provided are methods of screening for an agent that treats or prevents osteoporosis in a subject. The methods comprise administering to the subject with or at risk of developing osteoporosis an agent to be screened and determining whether the agent inhibits the level of expression or activity of an EP1 receptor. A decrease in the level of expression or activity of the EP1 receptor as compared to a control indicates the agent treats or prevents osteoporosis. The agent can, for example, be selected from the group consisting of a small molecule, a polypeptide, a peptidomimetic, or a combination thereof. Optionally, the level of expression of RNA encoding the EP1 receptor is determined. Optionally, the level of expression of the EP1 receptor protein is determined. Optionally, the level of activity of the EP1 receptor is determined.

A decrease or lower level of expression of the EP1 receptor, marker of osteoblast differentiation, or marker of osteoclast differentiation as compared to a control means that the level of expression of the EP1 receptor, marker of osteoblast differentiation, or marker of osteoclast differentiation is lower in the experimental sample being tested than in the control. An increase or higher level of expression of the EP1 receptor, marker of osteoblast differentiation, or marker of osteoclast differentiation as compared to a control means the level of expression of the EP1 receptor, marker of osteoblast differentiation, or marker of osteoclast differentiation is higher in the experimental sample being tested than in the control. Such differences between the test and control samples or subjects are optionally statistical differences or are at least one to two standard deviations of difference. Optionally, the difference is a level of at least 1.5× background. As used herein, control refers to an untreated sample, which includes a sample before or after any treatment effect has dissipated. Thus, the untreated sample can be from the same or different subject. The untreated sample can also be a cell from the same cell line or sample as the test cell or sample to be screened. The untreated sample can be used to create a baseline level of expression or activity in the subject or cell.

The level of expression of the EP1 receptor can, for example, be determined by detecting a level of EP1 receptor protein or a level of RNA encoding the EP1 receptor. The level of protein expression is determined using an assay selected from the group consisting of Western blot, enzyme-linked immunosorbent assay (ELISA), enzyme immunoassay (EIA), radioimmunoassay (RIA), or protein array. The level of RNA expression is determined using an assay selected from the group consisting of microarray analysis, gene chip, Northern blot, in situ hybridization, reverse transcription-polymerase chain reaction (RT-PCR), one step PCR, and quantitative real time (qRT)-PCR. The analytical techniques to determine protein or RNA expression are known. See, e.g. Sambrook et al., Molecular Cloning: A Laboratory Manual, 3rd Ed., Cold Spring Harbor Press, Cold Spring Harbor, N.Y. (2001).

The level of activity of the EP1 receptor can, for example, be determined by determining the regulation of osteoblast or osteoclast differentiation or function. An increase in osteoblast differentiation or function or a decrease in osteoclast differentiation or function as compared to a control indicates a decrease in activity of the EP1 receptor. The regulation of osteoblast or osteoclast differentiation or function can be determined by the level of one or more markers of osteoblast or osteoclast differentiation or function. A marker of osteoblast differentiation can be selected from the group consisting of alkaline phosphatase, runx2, osterix, osteocalcin, bone sialoprotein, and type 1 collagen. Markers of osteoclast differentiation or function can be selected from the group consisting of tartrate resistant acid phosphatase (TRAP), cathepsin K, and calcitonin receptor.

As used herein, an EP1 receptor inhibitory nucleic acid sequence can be a short-interfering RNA (siRNA) sequence or a micro-RNA (miRNA) sequence. A 21-25 nucleotide siRNA or miRNA sequence can, for example, be produced from an expression vector by transcription of a short-hairpin RNA (shRNA) sequence, a 60-80 nucleotide precursor sequence, which is subsequently processed by the cellular RNAi machinery to produce either a siRNA or miRNA sequence. Alternatively, a 21-25 nucleotide siRNA or miRNA sequence can, for example, be synthesized chemically. Chemical synthesis of siRNA or miRNA sequences is commercially available from such corporations as Dharmacon, Inc. (Lafayette, Colo.), Qiagen (Valencia, Calif.), and Ambion (Austin, Tex.). A siRNA sequence preferably binds a unique sequence within the EP1 receptor mRNA with exact complementarity and results in the degradation of the EP1 receptor mRNA molecule. A miRNA sequence preferably binds a unique sequence within the EP1 receptor mRNA with exact or less than exact complementarity and results in the translational repression of the EP1 receptor mRNA molecule. A miRNA sequence can bind anywhere within the EP1 receptor mRNA sequence, but preferably binds within the 3′ untranslated region of the EP1 receptor mRNA molecule. Methods of delivering siRNA or miRNA molecules are known in the art. See, e.g., Oh and Park, Adv. Drug. Deliv. Rev. 61(10):850-62 (2009); Gondi and Rao, J. Cell Physiol. 220(2):285-91 (2009); and Whitehead et al., Nat. Rev. Drug. Discov. 8(2):129-38 (2009).

As used herein, an EP1 receptor inhibitory nucleic acid sequence can be an antisense nucleic acid sequence. Antisense nucleic acid sequences can, for example, be transcribed from an expression vector to produce an RNA which is complementary to at least a unique portion of the EP1 receptor mRNA and/or the endogenous gene which encodes the EP1 receptor. Hybridization of an antisense nucleic acid under specific cellular conditions results in inhibition of EP1 receptor protein expression by inhibiting transcription and/or translation.

Antibodies described herein bind the EP1 receptor and antagonize the function of the EP1 receptor. The term antibody is used herein in a broad sense and includes both polyclonal and monoclonal antibodies. The term can also refer to a human antibody and/or a humanized antibody. Examples of techniques for human monoclonal antibody production include those described by Cole et al. (Monoclonal Antibodies and Cancer Therapy, Alan R. Liss, p. 77, 1985) and by Boerner et al. (J. Immunol. 147(1):86-95 (1991)). Human antibodies (and fragments thereof) can also be produced using phage display libraries (Hoogenboom et al., J. Mol. Biol. 227:381 (1991); Marks et al., J. Mol. Biol. 222:581 (1991)). The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA 90:2551-5 (1993); Jakobovits et al., Nature 362:255-8 (1993); Bruggermann et al., Year in Immunol. 7:33 (1993)).

As used herein, the term antibody encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (κ) and lambda (λ), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called alpha, delta, epsilon, gamma, and mu, respectively.

The term variable is used herein to describe certain portions of the antibody domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies. The constant domains are not involved directly in binding an antibody to an antigen, but exhibit various effector functions, such as participation of the antibody in antibody-dependent cellular toxicity.

As used herein, the term epitope is meant to include any determinant capable of specific interaction with the provided antibodies. Epitopic determinants usually consist of chemically active surface groupings of molecules such as amino acids or sugar side chains and usually have specific three dimensional structural characteristics, as well as specific charge characteristics. Identification of the epitope that the antibody recognizes is performed as follows. First, various partial structures of the target molecule that the monoclonal antibody recognizes are prepared. The partial structures are prepared by preparing partial peptides of the molecule. Such peptides are prepared by, for example, known oligopeptide synthesis technique or by incorporating DNA encoding the desired partial polypeptide in a suitable expression plasmid. The expression plasmid is delivered to a suitable host, such as E. coli, to produce the peptides. For example, a series of polypeptides having appropriately reduced lengths, working from the C- or N-terminus of the target molecule, can be prepared by established genetic engineering techniques. By establishing which fragments react with the antibody, the epitope region is identified. The epitope is more closely identified by synthesizing a variety of smaller peptides or mutants of the peptides using established oligopeptide synthesis techniques. The smaller peptides are used, for example, in a competitive inhibition assay to determine whether a specific peptide interferes with binding of the antibody to the target molecule. If so, the peptide is the epitope to which the antibody. binds. Commercially available kits, such as the SPOTs Kit (Genosys Biotechnologies, Inc., The Woodlands, Tex.) and a series of multipin peptide synthesis kits based on the multipin synthesis method (Chiron Corporation, Emeryvile, Calif.) may be used to obtain a large variety of oligopeptides.

The term antibody or fragments thereof can also encompass chimeric antibodies and hybrid antibodies, with dual or multiple antigen or epitope specificities, and fragments, such as F(ab′)2, Fab′, Fab and the like, including hybrid fragments. Thus, fragments of the antibodies that retain the ability to bind their specific antigens are provided. For example, fragments of antibodies which maintain EP1 receptor binding activity are included within the meaning of the term antibody or fragment thereof. Such antibodies and fragments can be made by techniques known in the art and can be screened for specificity and activity according to general methods for producing antibodies and screening antibodies for specificity and activity (See Harlow and Lane. Antibodies, A Laboratory Manual. Cold Spring Harbor Publications, New York (1988)).

Also included within the meaning of antibody or fragments thereof are conjugates of antibody fragments and antigen binding proteins (single chain antibodies) as described, for example, in U.S. Pat. No. 4,704,692, the contents of which are hereby incorporated by reference in their entirety.

Optionally, the antibody is a monoclonal antibody. The term monoclonal antibody as used herein refers to an antibody from a substantially homogeneous population of antibodies, i.e., the individual antibodies comprising the population are identical except for possible naturally occurring mutations that may be present in minor amounts. Monoclonal antibodies may be prepared using hybridoma methods, such as those described by Kohler and Milstein, Nature, 256:495 (1975) or Harlow and Lane, Antibodies, A Laboratory Manual. Cold Spring Harbor Publications, New York (1988). In a hybridoma method, a mouse or other appropriate host animal, is typically immunized with an immunizing agent to elicit lymphocytes that produce or are capable of producing antibodies that will specifically bind to the immunizing agent. Alternatively, the lymphocytes may be immunized in vitro. The immunizing agent can be the EP1 receptor or an immunogenic fragment thereof.

Generally, either peripheral blood lymphocytes (PBLs) are used in methods of producing monoclonal antibodies if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, pp. 59-103 (1986)). Immortalized cell lines are usually transformed mammalian cells, including myeloma cells of rodent, bovine, equine, and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”) substances that prevent the growth of HGPRT-deficient cells.

Immortalized cell lines useful here are those that fuse efficiently, support stable high level expression of antibody by the selected antibody-producing cells, and are sensitive to a medium such as HAT medium. Immortalized cell lines include murine myeloma lines, which can be obtained, for instance, from the Salk Institute Cell Distribution Center; San Diego, Calif. and the American Type Culture Collection; Rockville, Md. Human myeloma and mouse-human heteromyeloma cell lines also have been described for the production of human monoclonal antibodies (Kozbor, J. Immunol. 133:3001 (1984); Brodeur et al., Monoclonal Antibody Production Techniques and Applications, Marcel Dekker, Inc., New York, pp. 51-63 (1987)).

The culture medium in which the hybridoma cells are cultured can then be assayed for the presence of monoclonal antibodies directed against the EP1 receptor or selected epitopes thereof. The binding specificity of monoclonal antibodies produced by the hybridoma cells can be determined by immunoprecipitation or by an in vitro binding assay, such as radioimmunoassay (RIA) or enzyme-linked immunoabsorbent assay (ELISA). Such techniques and assays are known in the art, and are described further in Harlow and Lane Antibodies, A Laboratory Manual, Cold Spring Harbor Publications, New York (1988).

After the desired hybridoma cells are identified, the clones may be subcloned by limiting dilution or FACS sorting procedures and grown by standard methods. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells can serve as a preferred source of such DNA. Once isolated, the DNA may be placed into expression vectors, which are then transfected into host cells such as simian COS cells, Chinese hamster ovary (CHO) cells, plasmacytoma cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide. Such a non-immunoglobulin polypeptide can be substituted for the constant domains of an antibody provided herein, or can be substituted for the variable domains of one antigen-combining site of an antibody to create a chimeric bivalent antibody comprising one antigen-combining site having specificity for the EP1 receptor and another antigen-combining site having specificity for a different antigen.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′)2 fragment that has two antigen combining sites and is still capable of cross-linking antigen.

The Fab fragments produced in the antibody digestion can also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Fab′-SH is the designation herein for Fab′ in which the cysteine residue(s) of the constant domains bear a free thiol group.

One method of producing proteins comprising the provided antibodies or polypeptides is to link two or more peptides or polypeptides together by protein chemistry techniques. For example, peptides or polypeptides can be chemically synthesized using currently available laboratory equipment using either Fmoc (9-fluorenylmethyl-oxycarbonyl) or Boc (tert-butyloxycarbonoyl) chemistry (Applied Biosystems, Inc.; Foster City, Calif.). Those of skill in the art readily appreciate that a peptide or polypeptide corresponding to the antibody provided herein, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group that is functionally blocked on the other fragment. By peptide condensation reactions, these two fragments can be covalently joined via a peptide bond at their carboxyl and amino termini, respectively, to form an antibody, or fragment thereof. (Grant GA (1992) Synthetic Peptides: A User Guide. W.H. Freeman and Co., N.Y. (1992); Bodansky and Trost, Ed. (1993) Principles of Peptide Synthesis. Springer Verlag Inc., NY). Alternatively, the peptide or polypeptide can by independently synthesized in vivo. Once isolated, these independent peptides or polypeptides may be linked to form an antibody or fragment thereof via similar peptide condensation reactions.

For example, enzymatic ligation of cloned or synthetic peptide segments can allow relatively short peptide fragments to be joined to produce larger peptide fragments, polypeptides or whole protein domains (Abrahmsen et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776 779 (1994)). The first step is the chemoselective reaction of an unprotected synthetic peptide a thioester with another unprotected peptide segment containing an amino terminal Cys residue to give a thioester linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini et al., FEBS Lett. 307:97-101 (1992); Clark et al., J. Biol. Chem. 269:16075 (1994); Clark et al., Biochemistry 30:3128 (1991); Rajarathnam et al., Biochemistry 33:6623-30 (1994)).

Alternatively, unprotected peptide segments can be chemically linked where the bond formed between the peptide segments as a result of the chemical ligation is an unnatural (non peptide) bond (Schnolzer et al., Science 256:221 (1992)). This technique has been used to synthesize analogs of protein domains as well as large amounts of relatively pure proteins with full biological activity (deLisle et al., Techniques in Protein Chemistry IV. Academic Press, New York, pp. 257-267 (1992)).

The provided polypeptide fragments can be recombinant proteins obtained by cloning nucleic acids encoding the polypeptide in an expression system capable of producing the polypeptide fragments thereof, such as a bacterial, adenovirus or baculovirus expression system. For example, one can determine the active domain of an antibody from a specific hybridoma that can cause a biological effect associated with the interaction of the antibody with the EP1-receptor. For example, amino acids found to not contribute to either the activity or the binding specificity or affinity of the antibody can be deleted without a loss in the respective activity.

The provided fragments, whether attached to other sequences, can also include insertions, deletions, substitutions, or other selected modifications of particular regions or specific amino acids residues, provided the activity of the fragment is not significantly altered or impaired compared to the nonmodified antibody or epitope. These modifications can provide for some additional property, such as to remove or add amino acids capable of disulfide bonding, to increase its bio longevity, to alter its secretory characteristics, and the like. In any case, the fragment can possess a bioactive property, such as binding activity, regulation of binding at the binding domain, and the like. Functional or active regions may be identified by mutagenesis of a specific region of the protein, followed by expression and testing of the expressed polypeptide. Such methods are readily apparent to a skilled practitioner in the art and can include site specific mutagenesis of the nucleic acid encoding the antigen. (Zoller et al., Nucl. Acids Res. 10:6487-500 (1982)).

Further provided herein is a humanized or human version of the antibody. Optionally, the antibody modulates the activity of the EP1 receptor by activating or inhibiting the EP1 receptor. Optionally, the humanized or human antibody comprises at least one complementarity determining region (CDR) of an antibody having the same epitope specificity as an antibody produced by the hybridoma cell line disclosed herein. For example, the antibody can comprise all CDRs of an antibody having the same epitope specificity as an antibody produced by the hybridoma cell line.

Optionally, the humanized or human antibody can comprise at least one residue of the framework region of the monoclonal antibody produced by a disclosed hybridoma cell line. Humanized and human antibodies can be made using methods known to a skilled artesian; for example, the human antibody can be produced using a germ-line mutant animal or by a phage display library.

Antibodies can also be generated in other species and humanized for administration to humans. Alternatively, fully human antibodies can also be made by immunizing a mouse or other species capable of making a fully human antibody (e.g., mice genetically modified to produce human antibodies) and screening clones that bind the EP1 receptor. See, e.g., Lonberg and Huszar, Int. Rev. Immunol, 13:65-93, (1995), which is incorporated herein by reference in its entirety for methods of producing fully human antibodies. As used herein, the term humanized and human in relation to antibodies, relate to any antibody which is expected to elicit a therapeutically tolerable weak immunogenic response in a human subject. Thus, the terms include fully humanized or fully human as well as partially humanized or partially human.

Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a CDR of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all or at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992)).

Generally, a humanized antibody has one or more amino acid residues introduced into it from a source that is non-human. These non-human amino acid residues are often referred to as import residues, which are typically taken from an import variable domain. Humanization can be essentially performed following the methods described in Jones et al., Nature 321:522-525 (1986); Riechmann et al., Nature 332:323-327 (1988); or Verhoeyen et al., Science 239:1534-1536 (1988), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The nucleotide sequences encoding the provided antibodies can be readily isolated and sequenced using conventional procedures (e.g., by using oligonucleotide probes that are capable of binding specifically to genes encoding the heavy and light chains of murine antibodies). These nucleotide sequences can also be modified, or humanized, for example, by substituting the coding sequence for human heavy and light chain constant domains in place of the homologous murine sequences (see, e.g., U.S. Pat. No. 4,816,567). The nucleotide sequences encoding any of the provided antibodies can be expressed in appropriate host cells. These include prokaryotic host cells including, but not limited to, E. coli, Bacillus subtilus, other enterobacteriaceae such as Salmonella typhimurium or Serratia marcesans, and various Pseudomonas species. Eukaryotic host cells can also be utilized. These include, but are not limited to, yeast cells (for example, Saccharomyces cerevisiae and Pichia pastoris), and mammalian cells such as VERO cells, HeLa cells, Chinese hamster ovary (CHO) cells, W138 cells, BHK cells, COS-7 cells, 293T cells and MDCK cells. The antibodies produced by these cells can be purified from the culture medium and assayed for binding, activity, specificity or any other property of the monoclonal antibodies by utilizing the methods set forth herein and standard in the art.

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, e.g., Jakobovits et al., Proc. Natl. Acad. Sci. USA 90:2551-55 (1993); Jakobovits et al., Nature 362:255-8 (1993); Bruggemann et al., Year in Immuno. 7:33 (1993)). Human antibodies can also be produced in phage display libraries (Hoogenboom et al., J. Mol. Biol. 227:381 (1991); Marks et al., J. Mol. Biol. 222:581 (1991)). The techniques of Cole et al. and Boerner et al. are also available for the preparation of human monoclonal antibodies (Cole et al., Monoclonal Antibodies and Cancer Therapy, Alan R. Liss, ed., p. 77 (1985); Boerner et al., J. Immunol. 147(1):86-95 (1991)).

Provided herein are methods of accelerating bone fracture healing or treating or preventing osteoporosis in a subject. Such methods optionally include identifying a subject with a bone fracture, with osteoporosis or at risk of developing osteoporosis using any method accepted by one of skill in the art. Such methods also include administering an effective amount of an EP1 receptor inhibitor comprising a small molecule, a polypeptide, a nucleic acid molecule, a peptidomimetic or a combination thereof. Optionally, the small molecules, polypeptides, nucleic acid molecules, and/or peptidomimetics are contained within a pharmaceutical composition.

Provided herein are compositions containing the provided small molecules, polypeptides, nucleic acid molecules, and/or peptidomimetics and a pharmaceutically acceptable carrier described herein. The herein provided compositions are suitable of administration in vitro or in vivo. By pharmaceutically acceptable carrier is meant a material that is not biologically or otherwise undesirable, i.e., the material is administered to a subject without causing undesirable biological effects or interacting in a deleterious manner with the other components of the pharmaceutical composition in which it is contained. The carrier is selected to minimize degradation of the active ingredient and to minimize adverse side effects in the subject.

Suitable carriers and their formulations are described in Remington: The Science and Practice of Pharmacy, 21^{st} Edition, David B. Troy, ed., Lippicott Williams & Wilkins (2005). Typically, an appropriate amount of a pharmaceutically-acceptable salt is used in the formulation to render the formulation isotonic. Examples of the pharmaceutically-acceptable carriers include, but are not limited to, sterile water, saline, buffered solutions like Ringer's solution, and dextrose solution. The pH of the solution is generally about 5 to about 8 or from about 7 to 7.5. Other carriers include sustained release preparations such as semipermeable matrices of solid hydrophobic polymers containing the immunogenic polypeptides. Matrices are in the form of shaped articles, e.g., films, liposomes, or microparticles. Certain carriers may be more preferable depending upon, for instance, the route of administration and concentration of composition being administered. Carriers are those suitable for administration of the agent, e.g., the small molecule, polypeptide, nucleic acid molecule, and/or peptidomimetic, to humans or other subjects.

The compositions are administered in a number of ways depending on whether local or systemic treatment is desired, and on the area to be treated. Local administration, e.g., during a surgical procedure, can be with use of a bioabsorbent gel or matrix impregnated with the composition or by flooding the surgical site with the composition. The compositions are administered via any of several routes of administration, including topically, orally, parenterally, intravenously, intra-articularly, intraperitoneally, intramuscularly, subcutaneously, intracavity, transdermally, intrahepatically, intracranially, nebulization/inhalation, or by installation via bronchoscopy.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives are optionally present such as, for example, antimicrobials, anti-oxidants, chelating agents, and inert gases and the like.

Formulations for topical administration include ointments, lotions, creams, gels, drops, suppositories, sprays, liquids, and powders. Conventional pharmaceutical carriers, aqueous, powder, or oily bases, thickeners and the like are optionally necessary or desirable.

Compositions for oral administration include powders or granules, suspension or solutions in water or non-aqueous media, capsules, sachets, or tables. Thickeners, flavorings, diluents, emulsifiers, dispersing aids or binders are optionally desirable.

Optionally, the nucleic acid molecule or polypeptide is administered by a vector comprising the nucleic acid molecule or a nucleic acid sequence encoding the polypeptide. There are a number of compositions and methods which can be used to deliver the nucleic acid molecules and/or polypeptides to cells, either in vitro or in vivo via, for example, expression vectors. These methods and compositions can largely be broken down into two classes: viral based delivery systems and non-viral based deliver systems. Such methods are well known in the art and readily adaptable for use with the compositions and methods described herein.

As used herein, plasmid or viral vectors are agents that transport the disclosed nucleic acids into the cell without degradation and include a promoter yielding expression of the nucleic acid molecule and/or polypeptide in the cells into which it is delivered. Viral vectors are, for example, Adenovirus, Adeno-associated virus, herpes virus, Vaccinia virus, Polio virus, Sindbis, and other RNA viruses, including these viruses with the HIV backbone. Also preferred are any viral families which share the properties of these viruses which make them suitable for use as vectors. Retroviral vectors, in general are described by Coffin et al., Retroviruses, Cold Spring Harbor Laboratory Press (1997), which is incorporated by reference herein for the vectors and methods of making them. The construction of replication-defective adenoviruses has been described (Berkner et al., J. Virol. 61:1213-20 (1987); Massie et al., Mol. Cell. Biol. 6:2872-83 (1986); Haj-Ahmad et al., J. Virol. 57:267-74 (1986); Davidson et al., J. Virol. 61:1226-39 (1987); Zhang et al., BioTechniques 15:868-72 (1993)). The benefit and the use of these viruses as vectors is that they are limited in the extent to which they can spread to other cell types, since they can replicate within an initial infected cell, but are unable to form new infections viral particles. Recombinant adenoviruses have been shown to achieve high efficiency after direct, in vivo delivery to airway epithelium, hepatocytes, vascular endothelium, CNS parenchyma, and a number of other tissue sites. Other useful systems include, for example, replicating and host-restricted non-replicating vaccinia virus vectors.

The provided polypeptides and/or nucleic acid molecules can be delivered via virus like particles. Virus like particles (VLPs) consist of viral protein(s) derived from the structural proteins of a virus. Methods for making and using virus like particles are described in, for example, Garcea and Gissmann, Current Opinion in Biotechnology 15:513-7 (2004).

The provided polypeptides can be delivered by subviral dense bodies (DBs). DBs transport proteins into target cells by membrane fusion. Methods for making and using DBs are described in, for example, Pepperl-Klindworth et al., Gene Therapy 10:278-84 (2003).

The provided polypeptides can be delivered by tegument aggregates. Methods for making and using tegument aggregates are described in International Publication No. WO 2006/110728.

Non-viral based delivery methods can include expression vectors comprising nucleic acid molecules and nucleic acid sequences encoding polypeptides, wherein the nucleic acids are operably linked to an expression control sequence. Suitable vector backbones include, for example, those routinely used in the art such as plasmids, artificial chromosomes, BACs, YACs, or PACs. Numerous vectors and expression systems are commercially available from such corporations as Novagen (Madison, Wis.), Clonetech (Palo Alto, Calif.), Stratagene (La Jolla, Calif.), and Invitrogen/Life Technologies (Carlsbad, Calif.). Vectors typically contain one or more regulatory regions. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns.

Preferred promoters controlling transcription from vectors in mammalian host cells may be obtained from various sources, for example, the genomes of viruses such as polyoma, Simian Virus 40 (SV40), adenovirus, retroviruses, hepatitis B virus, and most preferably cytomegalovirus (CMV), or from heterologous mammalian promoters, e.g. β-actin promoter or EF1α promoter, or from hybrid or chimeric promoters (e.g., CMV promoter fused to the β-actin promoter). Of course, promoters from the host cell or related species are also useful herein.

Enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 base pairs (bp) in length, and they function in cis. Enhancers usually function to increase transcription from nearby promoters. Enhancers can also contain response elements that mediate the regulation of transcription. While many enhancer sequences are known from mammalian genes (globin, elastase, albumin, fetoprotein, and insulin), typically one will use an enhancer from a eukaryotic cell virus for general expression. Preferred examples are the SV40 enhancer on the late side of the replication origin, the cytomegalovirus early promoter enhancer, the polyoma enhancer on the late side of the replication origin, and adenovirus enhancers.

The promoter and/or the enhancer can be inducible (e.g. chemically or physically regulated). A chemically regulated promoter and/or enhancer can, for example, be regulated by the presence of alcohol, tetracycline, a steroid, or a metal. A physically regulated promoter and/or enhancer can, for example, be regulated by environmental factors, such as temperature and light. Optionally, the promoter and/or enhancer region can act as a constitutive promoter and/or enhancer to maximize the expression of the region of the transcription unit to be transcribed. In certain vectors, the promoter and/or enhancer region can be active in a cell type specific manner. Optionally, in certain vectors, the promoter and/or enhancer region can be active in all eukaryotic cells, independent of cell type. Preferred promoters of this type are the CMV promoter, the SV40 promoter, the β-actin promoter, the EF1α promoter, and the retroviral long terminal repeat (LTR).

The vectors also can include, for example, origins of replication and/or markers. A marker gene can confer a selectable phenotype, e.g., antibiotic resistance, on a cell. The marker product is used to determine if the vector has been delivered to the cell and once delivered is being expressed. Examples of selectable markers for mammalian cells are dihydrofolate reductase (DHFR), thymidine kinase, neomycin, neomycin analog G418, hygromycin, puromycin, and blasticidin. When such selectable markers are successfully transferred into a mammalian host cell, the transformed mammalian host cell can survive if placed under selective pressure. Examples of other markers include, for example, the E. coli lacZ gene, green fluorescent protein (GFP), and luciferase. In addition, an expression vector can include a tag sequence designed to facilitate manipulation or detection (e.g., purification or localization) of the expressed polypeptide. Tag sequences, such as GFP, glutathione S-transferase (GST), polyhistidine, c-myc, hemagglutinin, or FLAG™ tag (Kodak; New Haven, Conn.) sequences typically are expressed as a fusion with the encoded polypeptide. Such tags can be inserted anywhere within the polypeptide including at either the carboxyl or amino terminus.

As used herein, the terms peptide, polypeptide, or protein are used broadly to mean two or more amino acids linked by a peptide bond. Protein, peptide, and polypeptide are also used herein interchangeably to refer to amino acid sequences. It should be recognized that the term polypeptide is not used herein to suggest a particular size or number of amino acids comprising the molecule and that a peptide of the invention can contain up to several amino acid residues or more.

As used throughout, subject can be a vertebrate, more specifically a mammal (e.g., a human, horse, cat, dog, cow, pig, sheep, goat, mouse, rabbit, rat, and guinea pig), birds, reptiles, amphibians, fish, and any other animal. The term does not denote a particular age or sex. Thus, adult and newborn subjects, whether male or female, are intended to be covered. As used herein, patient or subject may be used interchangeably and can refer to a subject with a disease or disorder (e.g., osteoporosis or bone fracture). The term patient or subject includes human and veterinary subjects.

A subject at risk of developing a disease or disorder can be genetically predisposed to the disease or disorder, e.g., have a family history or have a mutation in a gene that causes the disease or disorder; be on medication that reduces bone density, e.g., steroids, or show early signs or symptoms of the disease or disorder. A subject currently with a disease or disorder has one or more than one symptom of the disease or disorder and may have been diagnosed with the disease or disorder.

The methods and agents as described herein are useful for both prophylactic and therapeutic treatment. For prophylactic use, a therapeutically effective amount of the agents described herein are administered to a subject prior to onset (e.g., before obvious signs of osteoporosis) or during early onset (e.g., upon initial signs and symptoms of osteoporosis). Prophylactic administration can occur for several days to years prior to the manifestation of symptoms of osteoarthritis or intervertebral disc disease. Prophylactic administration can be used, for example, in the preventative treatment of subjects diagnosed with a genetic predisposition to osteoarthritis or intervertebral disc disease or after joint surgery or trauma. Therapeutic treatment involves administering to a subject a therapeutically effective amount of the agents described herein after diagnosis or development of osteoporosis or bone fracture.

According to the methods taught herein, the subject is administered an effective amount of the agent. The terms effective amount and effective dosage are used interchangeably. The term effective amount is defined as any amount necessary to produce a desired physiologic response. Effective amounts and schedules for administering the agent may be determined empirically, and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary, and can be administered in one or more dose administrations daily, for one or several days. Guidance can be found in the literature for appropriate dosages for given classes of pharmaceutical products.

As used herein the terms treatment, treat, or treating refers to a method of reducing the effects of a disease or condition or symptom of the disease or condition. Thus in the disclosed method, treatment can refer to a 10%, 20%, 30%, 40%, 50%, 60%, 70%, 80%, 90%, or 100% reduction in the severity of an established disease or condition or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

As used herein, the terms prevent, preventing, and prevention of a disease or disorder refers to an action, for example, administration of a therapeutic agent, that occurs before or at about the same time a subject begins to show one or more symptoms of the disease or disorder, which inhibits or delays onset or exacerbation of one or more symptoms of the disease or disorder. As used herein, references to decreasing, reducing, or inhibiting include a change of 10%, 20%, 30%, 40%, 50%, 60%, 70%, 80%, 90% or greater as compared to a control level. Such terms can include but do not necessarily include complete elimination.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

### Materials and Methods

### Experimental Animals.

Wild-type (WT) C57BL/6J mice were purchased from Jackson Laboratories (Bar Harbor, Me.). The EP1^{-/-} mice (C57BL/6J background) were generously provided Dr. Matthew D. Breyer of Vanderbilt University (Nashville, Tenn.).

### Femoral Fracture Model.

Closed femur fractures were created in 10-week-old EP1^{-/-} mice and WT C57BL/6J. Mice were anesthetized with 60 mg/kg ketamine and 4 mg/kg xylazine intraperitoneally (i.p.). A 23 G needle (BD Medical System; Franklin Lakes, N.J.) was inserted into the length of medullary canal of the femur from the distal end, and a mid-diaphyseal fracture was created via three-point bending with an Einhorn device as previously described (Bonnarens and Einhorn, J. Orthop. Res. 2:97-101 (1984)). Healing of the fracture femur was monitored using radiographs, which were obtained at 0, 7, 14, 21 days under anesthesia using a Faxitron x-ray system (Faxitron x-ray; Lincolnshire, Ill.).

### Histology and Analysis.

The fractured femurs were collected at day 5, 7, 10, 14, 21, 28 and 35 post-fracture. Excess muscle and soft tissue was excised. Four specimens in each group were fixed in 10% neutral buffered formalin. The specimens were decalcified for 21 days in 14% EDTA (pH 7.2), embedded in paraffin, and sectioned at a thickness of 3 μm. Levels were cut at depths of 30 μm for histomorphometric analysis (3 levels per animal). The sections were stained using alcian blue hematoxylin/orange G eosin (ABH/OGE) and cytochemically for TRAcP. Total callus area, cartilage area, and woven bone area were quantified using a standardized eyepiece grid as previously described (Naik et al., J. Bone Miner. Res. 24:251-64 (2009)). Immunohistochemistry was also performed on these sections using a previously described method (Mungo et al., J. Bone Miner. Res. 20:159-62 (2002)). Mouse primary EP4 antibody (Cayman Chemical; Ann Arbor, Mich.) was used at a 1:200 dilution.

### Bone Marrow Cell Culture.

Bone marrow cells were isolated from 10-week-old EP1^{-/-} and C57BL/6J mice. Cells were cultured in 2 ml α-MEM containing 10% FBS at 5×10⁶ cells/well in 6-well plates. After being cultured for 7 days, the medium was replaced with media containing beta-glycerophosphate and ascorbic acid, and the media was changed every two days thereafter. Cells were harvested on day 10, 12, 14, 17 and 21 after plating for alkaline phosphatase and alizarin red staining, and mRNA analysis by quantitative real-time PCR. Cell proliferation was examined using the Cell Proliferation ELISA BrdU (colorimetric) immunoassay kit (Roche; Indianapolis, Ind.) according to the manufacturer's instructions. Cell viability was examined using the CellTiter-Blue Cell Viability Assay (Promega; Madison, Wis.) according to the manufacturer's instructions. Intracellular cAMP activity in bone marrow cells was examined using the cAMP-Glo Assay (Promega) according to the manufacturer's instructions.

cDNA Synthesis and Quantitative Real-Time PCR.

Mice were sacrificed post fracture at 3, 7, 10, 14, 21 and 28 days (N=4). Fracture callus tissue samples were carefully dissected from soft tissue and homogenized using the TissueLyzer (Qiagen; Valencia, Calif.). Total RNA was then extracted from homogenized samples by the RNeasy Fibrous Tissue Midi Kit (Qiagen) according to the manufacturer's instructions. One microgram aliquots of RNA were reverse transcribed into cDNA using the iScript cDNA Synthesis Kit (Bio-Rad; Hercules, Calif.). Real-time KR was performed using the Rotor Gene 6000 real-time DNA amplification system (Qiagen) according to the manufacturer's instruction. Realtime-PCR analyses were performed using murine specific primers for β-actin, col1a1, col2a1, col10a1, osteocalcin, ALP, Runx2, Osterix, RANKL, OPG, EP1, and fibronectin expression (Table 1).

TABLE 1
List of oligonucleotide primer sequences
for Real-Time PCR

| Target | Sequence |
|---|---|
| β-actin | 5′-AGATGTGGATCAGCAAGCAG-3′ |
| | (SEQ ID NO: 1) |
| | 5′-GCGCAAGTTAGGTTTTGTCA-3′ |
| | (SEQ ID NO: 2) |
| Col1a1 | 5′-GCCAAGGCAACAGTCGCT-3′ |
| | (SEQ ID NO: 3) |
| | 5′-CTTGGTGGTTTTGTATTCGATGAC-3′ |
| | (SEQ ID NO: 4) |
| Col2a1 | 5′-ACTGGTAAGTGGGGCAAGAC-3′ |
| | (SEQ ID NO: 5) |
| | 5′-CCACACCAAATTCCTGTTCA-3′ |
| | (SEQ ID NO: 6) |
| Col10a1 | 5′-CTTTGTGTGCCTTTCAATCG-3′ |
| | (SEQ ID NO: 7) |
| | 5′-GTGAGGTACAGCCTACCAGTTTT-3′ |
| | (SEQ ID NO: 8) |
| Osteocalcin | 5′-AGGGAGGATCAAGTCCCG-3′ |
| | (SEQ ID NO: 9) |
| | 5′-GAACAGACTCCGGCGCTA-3′ |
| | (SEQ ID NO: 10) |
| ALP | 5′-TGACCTTCTCTCCTCCATCC-3′ |
| | (SEQ ID NO: 11) |
| | 5′-CTTCCTGGGAGTCTCATCCT-3′ |
| | (SEQ ID NO: 12) |
| Runx2 | 5′-GCCGGGAATGATGAGAACTA-3′ |
| | (SEQ ID NO: 13) |
| | 5′-GGACCGTCCACTGTCACTTT-3′ |
| | (SEQ ID NO: 14) |
| Osterix | 5′-GTCAAGAGTCTTAGCCAAACTC-3′ |
| | (SEQ ID NO: 15) |
| | 5′-AAATGATGTGAGGCCAGATGG-3′ |
| | (SEQ ID NO: 16) |
| RANKL | 5′-CACCATCAGCTGAAGATAGT-3′ |
| | (SEQ ID NO: 17) |
| | 5′-CCAAGATCTCTAACATGACG-3′ |
| | (SEQ ID NO: 18) |
| OPG | 5′-AGTCCGTGAAGCAGGAGTG-3′ |
| | (SEQ ID NO: 19) |
| | 5′-CCATCTGGACATTTTTTGCAAA-3′ |
| | (SEQ ID NO: 20) |
| EP1 | 5′-TACATGGGATGCTCGAAACA-3′ |
| | (SEQ ID NO: 23) |
| | 5′-TTTTAAGCCCGTGTGGGTAG-3′ |
| | (SEQ ID NO: 24) |
| Fibronectin | 5′-GTGAAGAACGAGGAGGATGTG-3′ |
| | (SEQ ID NO: 25) |
| | 5′-GTGATGGCGGATGATGTAGC-3′ |
| | (SEQ ID NO: 26) |

### Spleen Cell Culture.

Spleen cells were isolated from 2-month-old EP1^{-/-} and C57BL/6J mice and cultured 50,000 cells/well in a-MEM containing 10% FBS, 10 ng/ml macrophage-colony stimulating factor (M-CSF; R&D Systems) and 50 ng/ml RANKL protein (R&D Systems; Minneapolis, Minn.) for 5 days as previously described (Wei et al., J. Bone Miner. Res. 20:1136-48 (2005)). The cells were fixed with formalin and stained for TRAcP activity. Multinucleated TRAcP+ cells were counted as osteoclasts.

### Western Blot Analysis.

Bone marrow cells were washed with cold phosphate-buffered saline (PBS) three times and lysed on ice in Golden Lysis Buffer (GLB) for 30 minutes as previously described (Li et al., Exp. Cell Res. 300:159-69 (2004)). The soluble and insoluble portions in the cell lysate were separated by centrifugation at 13,000×g for 30 minutes. The insoluble portion was discarded and the protein concentration of the soluble portion was examined using Coomassie Plus Protein Assay kit (Pierce Biotechnology; Rockford, Ill.). 15 μg aliquots of protein extract were separated by SDS-PAGE. After transfer to a PVDF membrane (Invitrogen; Carlsbad, Calif.), the blots were probed with the following antibodies: anti-EP2 receptor, anti-EP4 receptor, and anti-β-actin (Sigma; St. Louis, Mo.) at a dilution of 1:1000. Alkaline phosphatase-conjugated goat anti-rabbit and goat anti-mouse antibodies (Pierce Biotechnology) were used as secondary antibodies. The immune complexes were detected using NBT/BCIP substrate (Pierce Biotechnology).

### Statistics.

Results are shown as the mean value±the standard error of the mean (SEM). Statistical significance was identified by Student t-tests and two-way ANOVA followed by Dunnet's test. P-values less than 0.05 were considered significant.

### Example 1

### Fractures in EP1^{-/-} Mice Undergo Accelerated Endochondral Bone Formation and Healing

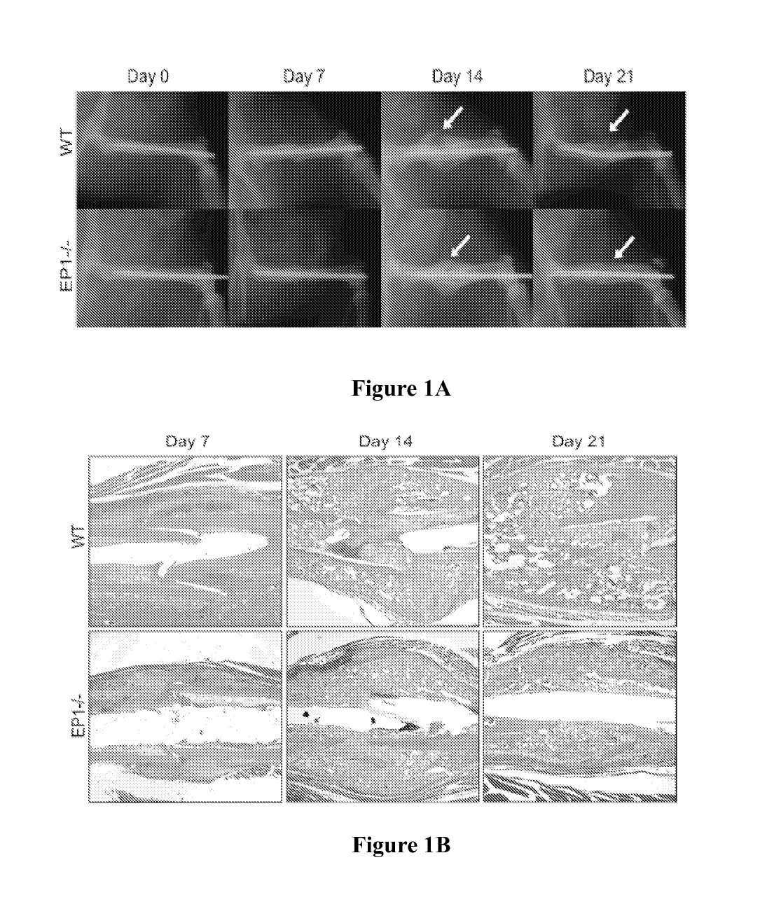

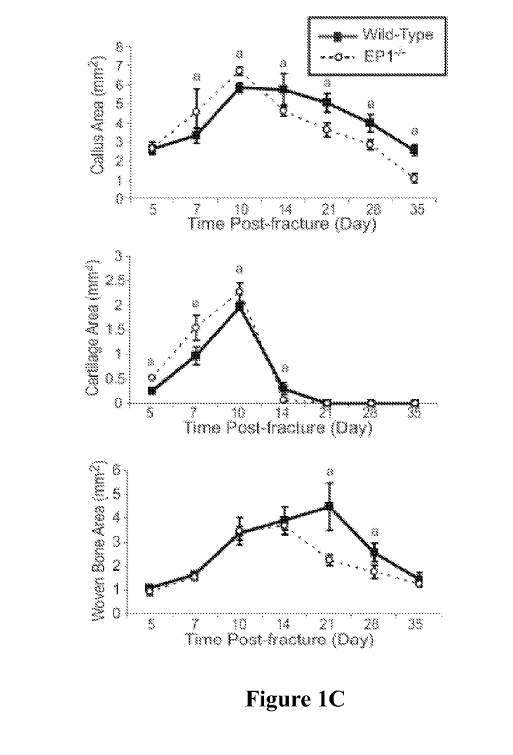

A murine femur fracture model was used to study fracture healing in the EP1^{-/-} mice. Fracture callus from 10-week-old EP1^{-/-} mice and age/sex-matched wild-type (WT) control mice was examined by radiographic analysis and histological staining at day 7, 14, and 21 following fracture. Both radiographs and histology demonstrate accelerated fracture healing in EP1^{-/-} mice compared to wild type mice (FIG. 1). At day 7 and 10 following fracture, EP1^{-/-} mice had increased callus area and more cartilage formation compared to wild type mice (FIGS. 1B and 1C). By day 14, fractures in EP1^{-/-} mice were healed and cartilage tissue was essentially absent in the callus. In contrast, healing was incomplete in 14-day-old fractures in wild type mice where a central area of cartilage tissue persisted (FIGS. 1A, 1B, and 1C). In addition to the accelerated mineralization, fractures in the EP1^{-/-} mice underwent more rapid remodeling. Callus area peaked at day 10 in EP1 knock out mice and then progressively decreased. In contrast peak callus area occurred at days 10 to 14 in wild type mice. Beginning at day 14, wild type mice had increased callus area and this persisted throughout the 35 day time course.

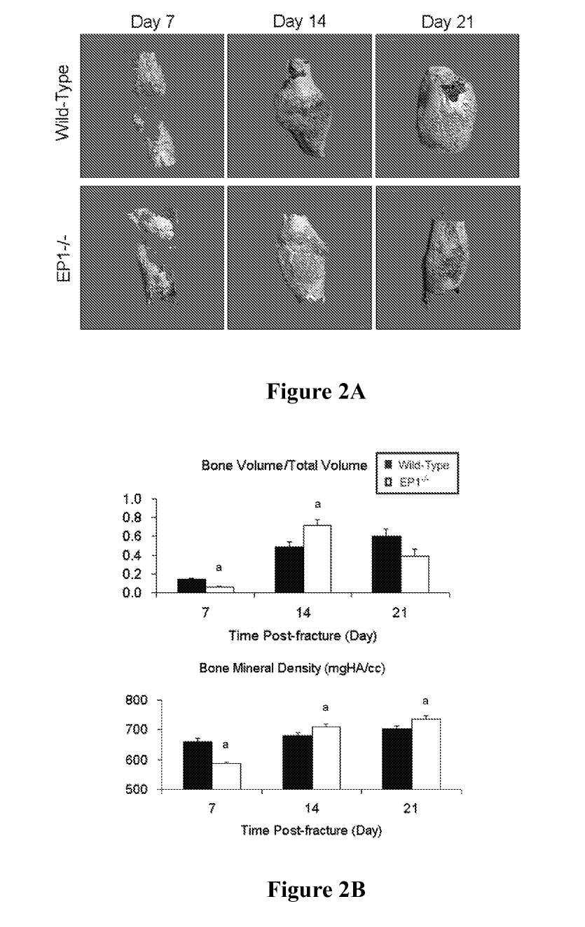

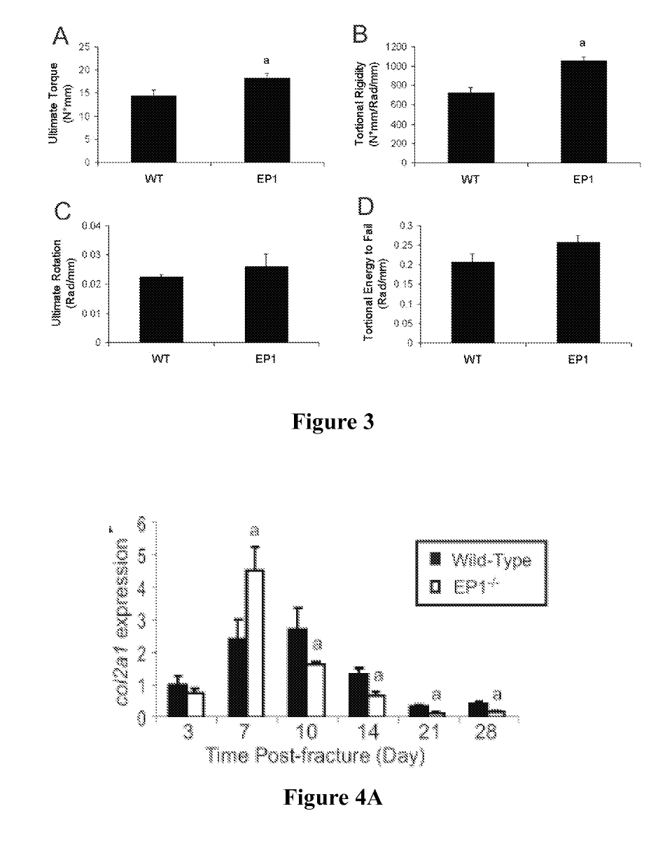

The mineralized calluses were examined by micro-computed tomography (μ-CT) analysis (FIG. 2). Consistent with the results from radiographic analysis and histological staining, 14-day-old fractures in the EP1^{-/-} mice were completely bridged by a calcified callus, while fractures in wild-type mice still exhibited a large gap (FIGS. 2A and 2B). At day 21 post-fracture, femurs in the EP1^{-/-} mice almost returned to normal shape compared with the large callus seen in wild-type mice. Reconstruction of the μ-CT data showed that fracture calluses from the EP1^{-/-} mice had higher bone mineral density than wild-type mice after 14 days of healing (FIG. 2B). Torsion testing was performed to examine the strength of the femurs from the EP1^{-/-} and wild-type control mice at day 21 post-fracture (FIG. 3). Femurs from the EP1^{-/-} mice had higher ultimate torque, torsional rigidity, ultimate rotation and torsional energy to fail.

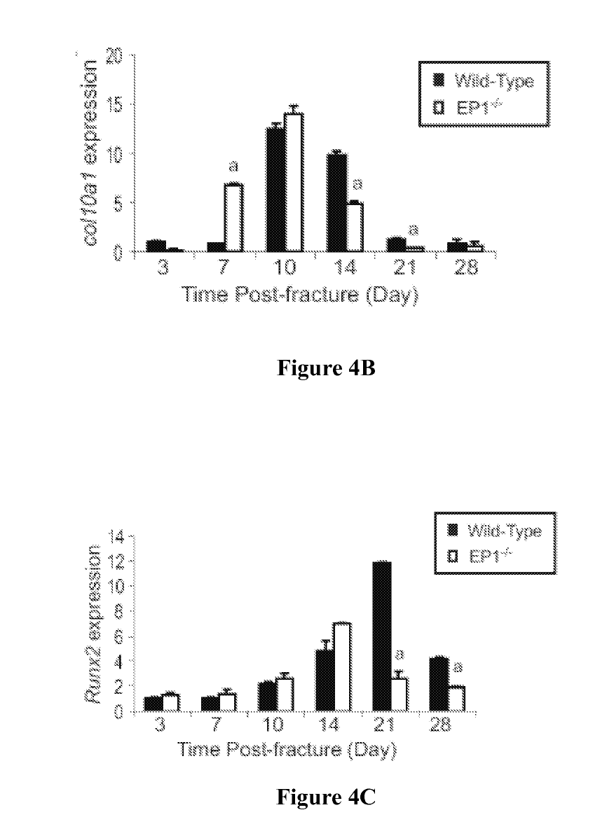

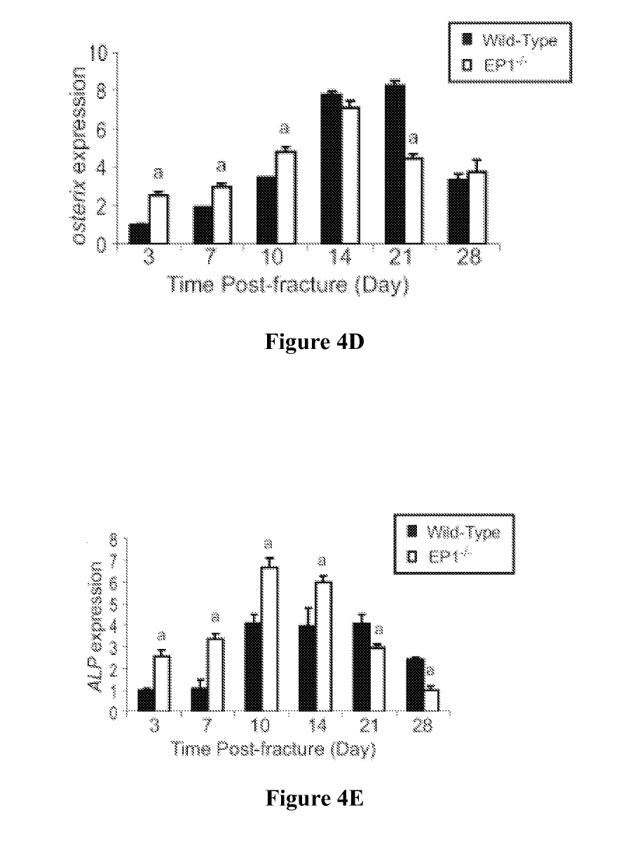

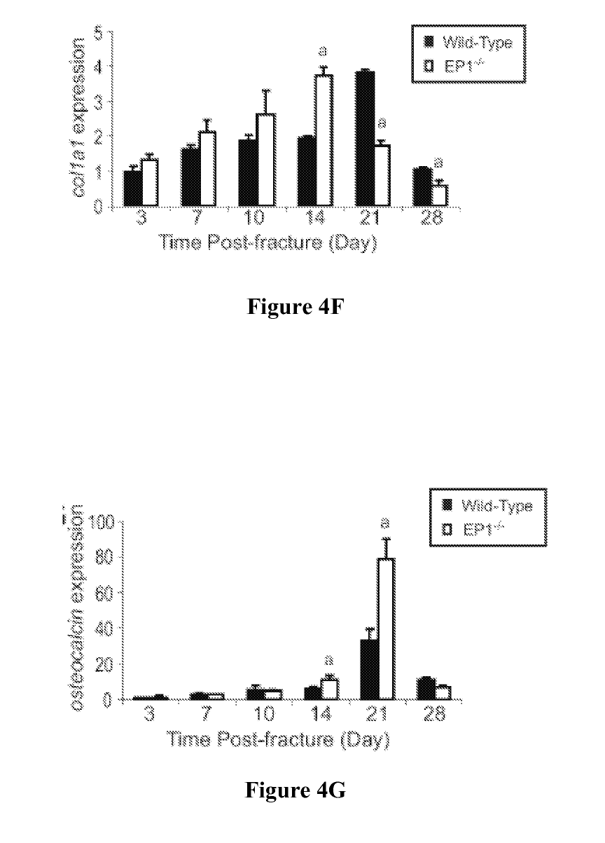

Quantitative real-time PCR analysis was performed to determine the expression of genes associated with endochondral bone formation in wild-type and EP1^{-/-} mice (FIGS. 4A and 4B). Both type 2 collagen (col2a1) and type 10 collagen (col10a1) expression were elevated earlier during fracture repair in the EP1^{-/-} mice compared to the wild type mice, so that expression levels of these genes were significantly higher in EP1^{-/-} mice at 7 days following fracture (FIGS. 4A and 4B). In contrast, the expression of col2a1 and col10a1 was elevated later in wild type fractures so that col2a1 and col10a1 were significantly increased in fractures from wild type mice compared to EP1^{-/-} mice between 14 and 21 days after fracture. The more rapid appearance of the cartilage specific genes and their earlier disappearance from fracture callus in EP1^{-/-} mice is consistent with accelerated endochondral bone formation compared to wild type mice.

### Example 2

### Osteoblast Differentiation is Accelerated in Fractures in EP1^{-/-} Mice

The expression of genes involved in osteoblast differentiation was also examined in the callus tissue of fractures from EP1^{-/-} and wild type mice (FIGS. 4C-4G). The expressions of the osteoblast specific transcription factors, Runx2 and osterix, were accelerated in fracture callus from EP1^{-/-} mice (FIGS. 4C and 4D). In mice, Runx2 and osterix expression peaked at day 14, while in wild-type callus tissue these genes had maximal expression at day 21 (FIGS. 4C and 4D). Expression of the osteoblast differentiation markers alkaline phosphatase (ALP), type I collagen (col1a1) and osteocalcin were also elevated earlier in fractures from EP1^{-/-} mice, consistent with accelerated osteoblast differentiation (FIGS. 4E-4G). Furthermore, the magnitude of both ALP and osteocalcin was higher in fractures in EP1^{-/-} mice. ALP expression in fractures from EP1^{-/-}. mice was significantly higher than wild-type from day 3 to day 14, with peak expression occurring 10 days after fracture. In contrast, ALP expression in fractures in wild type mice had a broad peak of maximal expression between 10 and 21 days, and the expression levels in fractures from wild type mice were increased compared to EP1^{-/-} mice at 21 and 28 days. The expression of col1a1 and osteocalcin were similarly elevated earlier in fractures in EP1^{-/-} mice compared to wild type mice. These findings suggest that osteoblast precursors undergo more rapid differentiation in EP1^{-/-} mice.

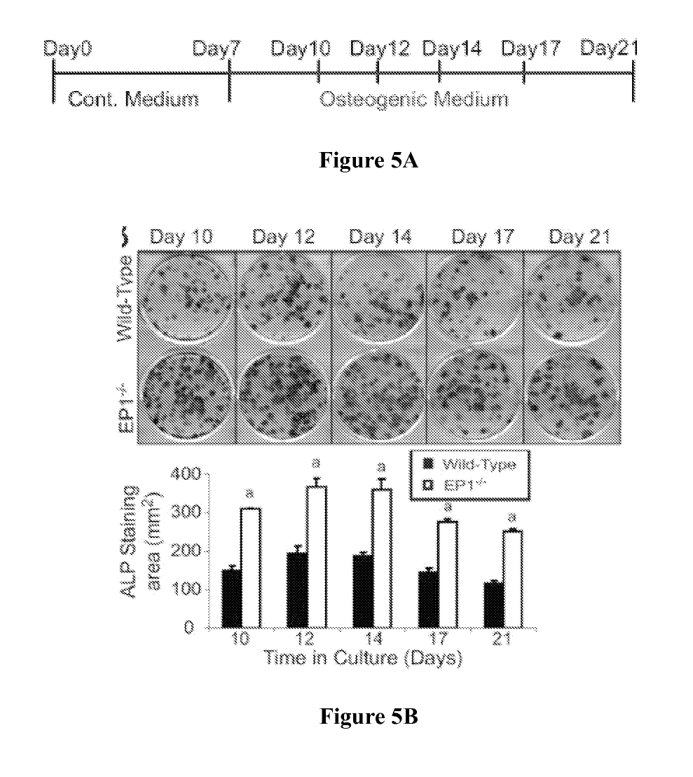

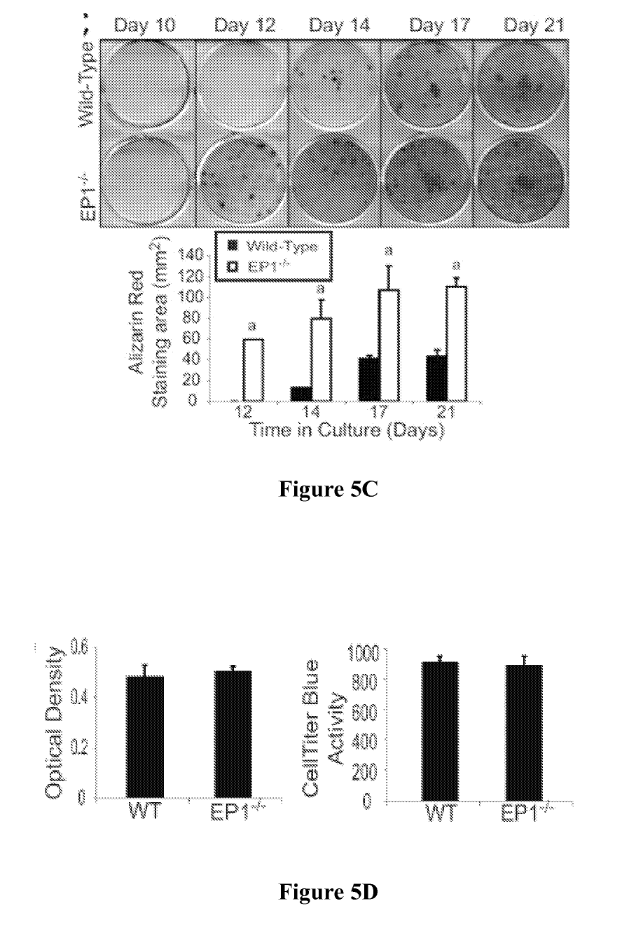

To determine whether mesenchymal stem cells from EP1^{-/-} mice have enhanced osteogenic potential, bone marrow stem cells extracted from EP1^{-/-} mice and wild type mice were isolated and placed in culture. After 7 days, beta-glycerol-phosphate (BGP) and ascorbic acid was added to the cultures and alkaline phosphatase activity and alizarin red staining were performed over time (FIG. 5A). Alkaline phosphatase staining showed more colonies formed with higher alkaline phosphatase activity in EP1^{-/-} bone marrow stem cell cultures (FIG. 5B). Similarly, Alizarin red staining was increased in EP1^{-/-} bone marrow stem cell cultures compared to control cultures, consistent with accelerated bone nodule formation (FIG. 5C). BrdU activity assay, performed at day 10, showed that EP1^{-/-} and wild type bone marrow cells have similar rates of proliferation (FIG. 5D). CellTiter blue activity confirmed that a similar number of viable cells were present in both cultures (FIG. 5D).

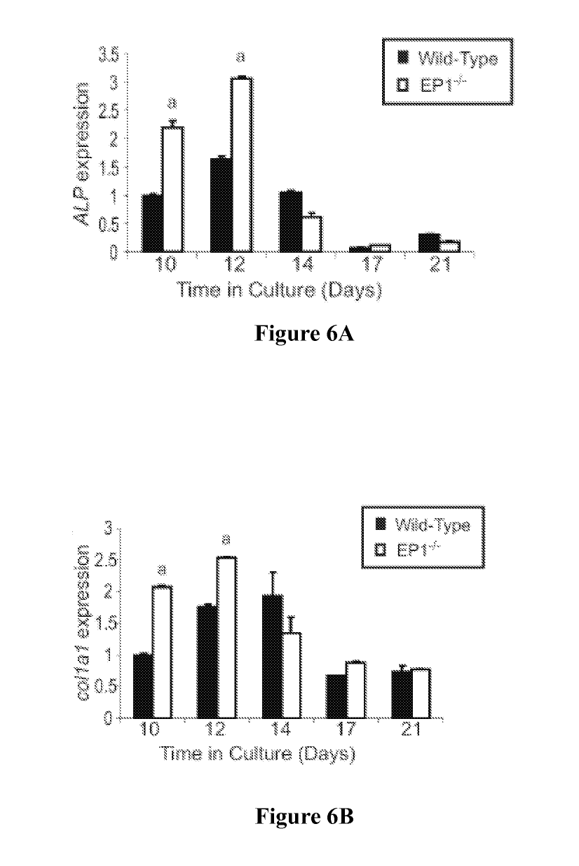

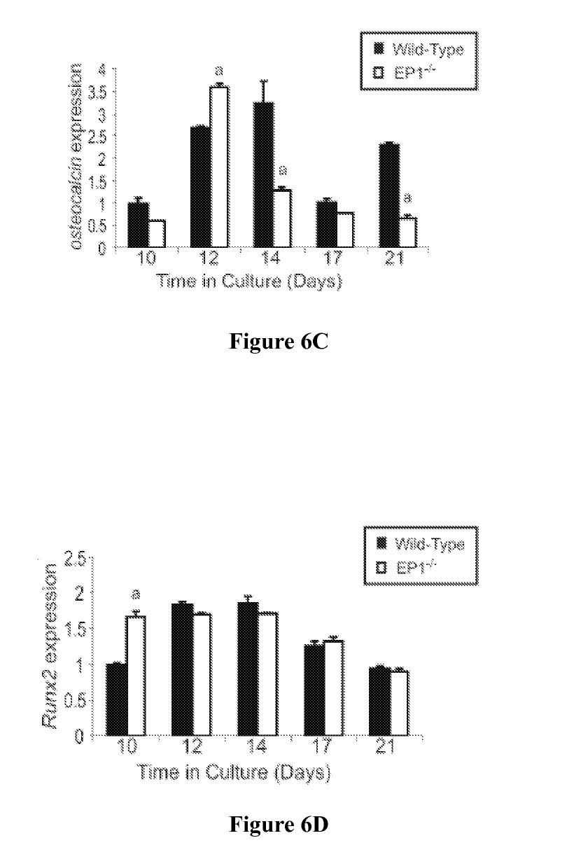

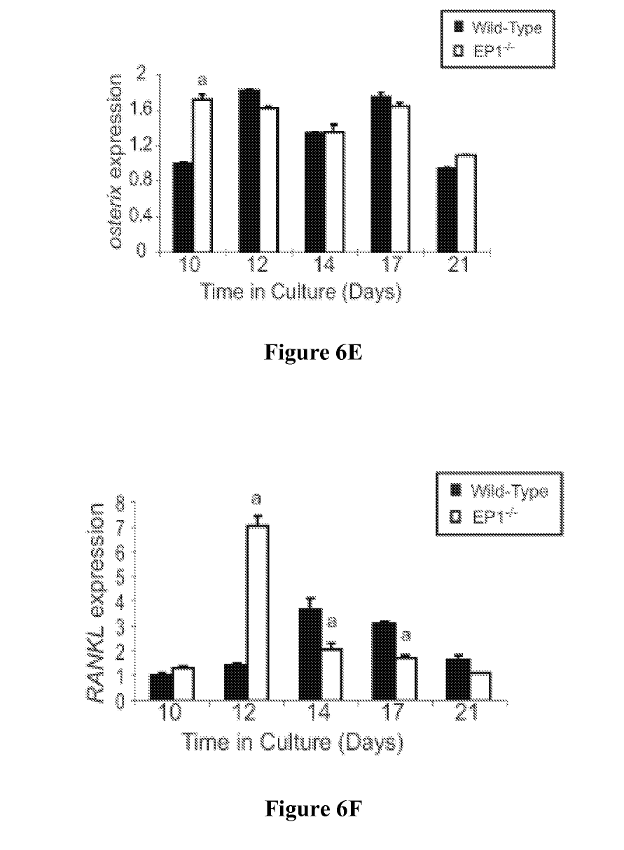

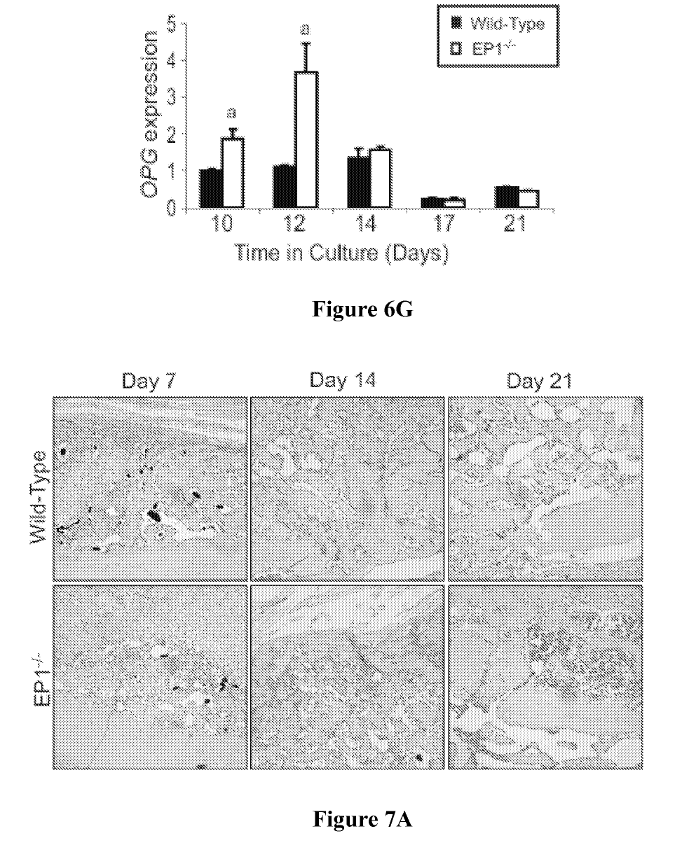

Total RNA was extracted from the bone marrow cultures to examine the expression of genes associated with osteoblast differentiation. EP1^{-/-} bone marrow stem cell cultures had enhanced early expression ALP, Col1a1, osteocalcin, Runx2 and osterix consistent with accelerated osteoblastogenesis compared to bone marrow cells from wild type mice (FIGS. 6A-6E). EP1^{-/-} bone marrow stem cell cultures also had increased expression of RANK ligand (RANKL) and osteoprotegerin (OPG) (FIGS. 6F and 6G).

### Example 3

### Fractures in EP1^{-/-} Mice have Accelerated Remodeling Due to Enhanced Osteoclast Inducing Signals

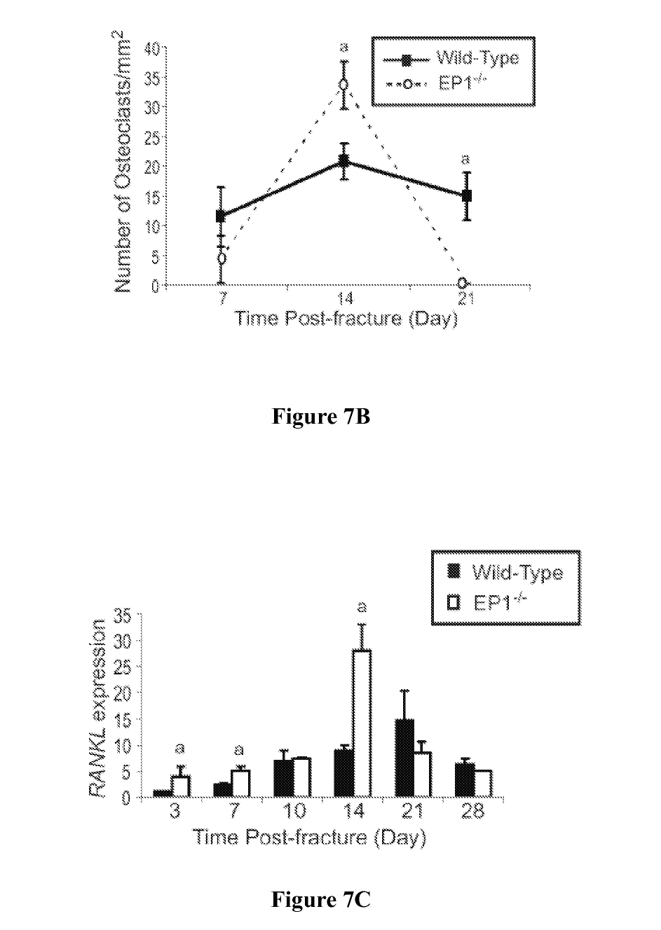

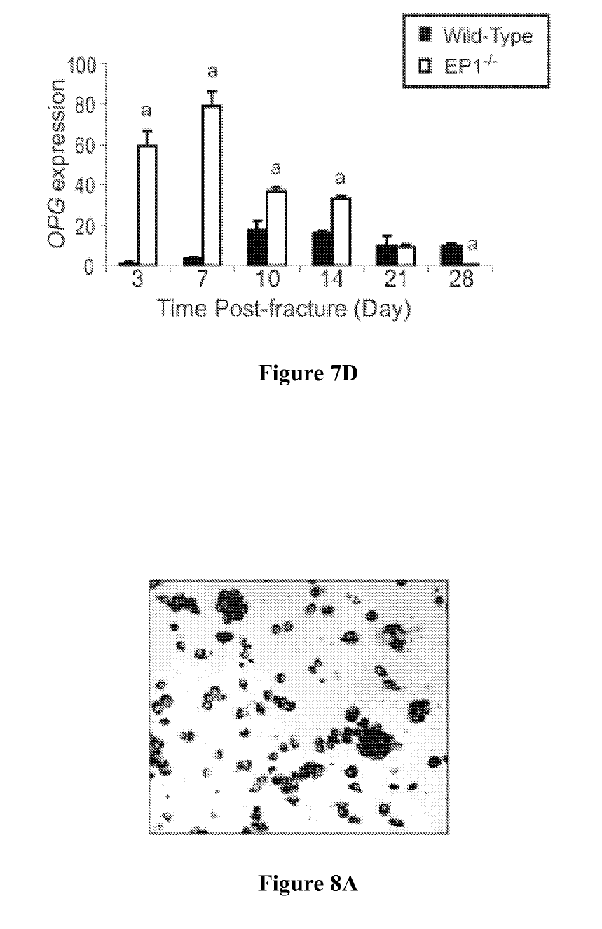

Since histology and radiographic examination suggested that the fractures in EP1^{-/-} mice underwent more rapid remodeling (FIG. 1), the fracture calluses were stained for the expression of TRAcP. The number of osteoclasts was increased 58% (p<0.05) in 14 day fracture callus from EP1^{-/-} mice compared to wild-type mice, consistent with the accelerated bone remodeling in fractures from EP1^{-/-} mice (FIG. 7A). By day 21, fractures in wild-type mice contained similar numbers of osteoclasts as that observed in the day 14 fractures from EP1^{-/-} mice. Surprisingly few osteoclasts were observed in 21 day old fractures from in EP1^{-/-} mice (FIGS. 7A and 7B). Total RNA was extracted from the calluses at various times and RT-PCR performed to measure the expression of osteoclast inducing genes, RANK ligand (RANKL) and its soluble receptor antagonist, osteoprotegerin (OPG). Fracture calluses from EP1^{-/-} mice had a higher overall level expression of both RANKL and OPG and both genes were elevated earlier in fractures in EP1^{-/-} mice compared to wild type mice (FIGS. 7C and 7D). The maximum expressions of RANKL occurred after 14 days in fractures in EP1^{-/-} mice and after 21 days in wild type mice. While RANKL had a sharp peak of expression in fractures in the EP1^{-/-} mice, the expression in wild type mice had a lower magnitude and was more sustained, consistent with the observed differences in osteoclast numbers in the fracture calluses.

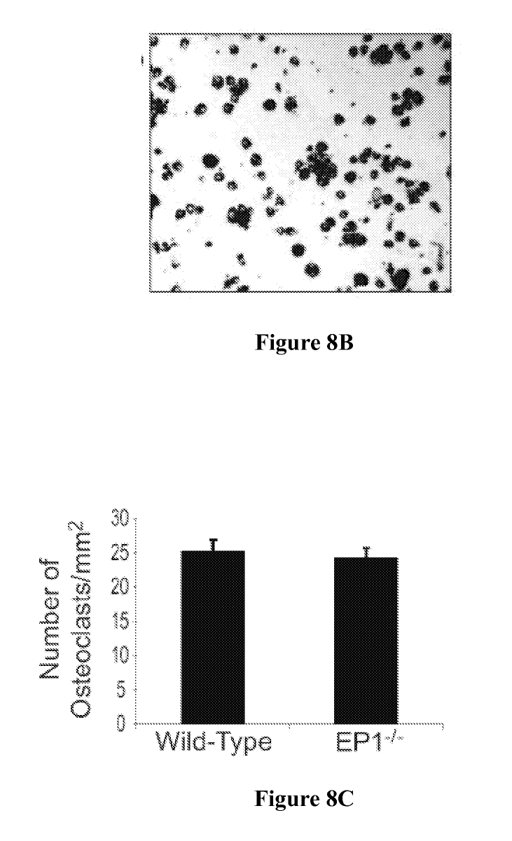

To examine if the accelerated osteoclast formation observed in EP1 fractures is due to a cell autonomous effect in osteoclast precursors in EP1^{-/-} mice, spleen cells were isolated from EP1^{-/-} and wild type mice and were placed in cell culture (FIG. 8). TRAcP staining performed at day 5 showed that similar numbers of osteoclasts formed in both EP1^{-/-} and wild-type cultures (FIG. 8). This result suggests that the accelerated osteoclast formation in the EP1^{-/-} fractures is primarily driven by signals from enhanced osteoblastogenesis.

### Example 4

### EP1^{-/-} Mice do not have Increased Expression or Activation of the EP2 or EP4 Receptors

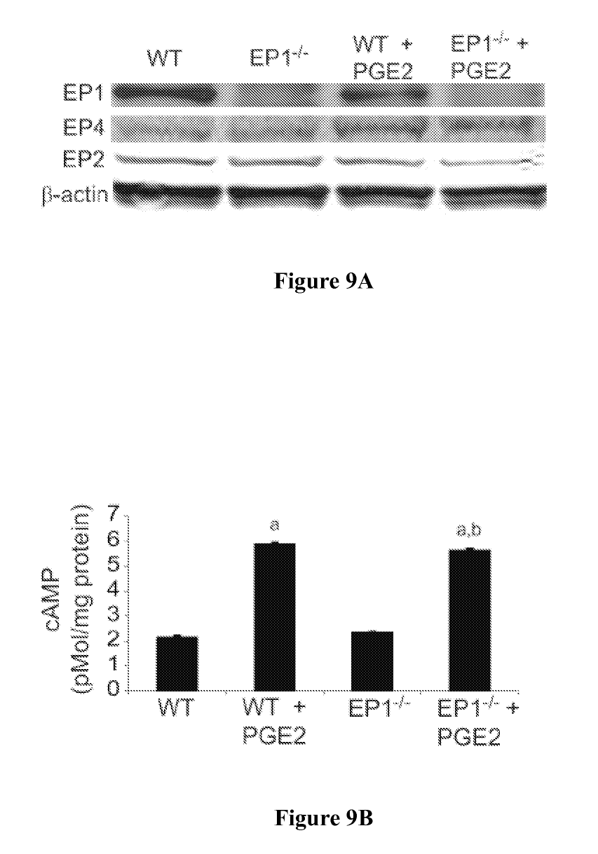

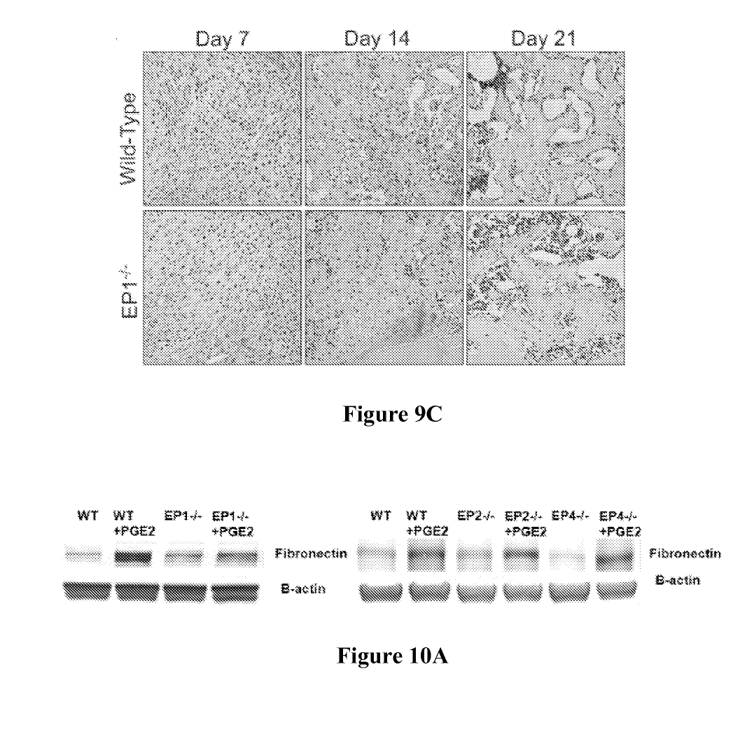

Since prior work has established that activation of the EP2 or EP4 receptors can accelerate fracture healing, experiments were performed to confirm that deletion of EP1 does not result in increased expression or activity of the EP2 and/or EP4 receptors. Protein levels of EP2 and EP4 receptors in EP1^{-/-} and wild type bone marrow cultures were examined. While EP1^{-/-} bone marrow cells had no expression of EP1, the expression of EP2 and EP4 was unchanged compared to bone marrow cells from wild type mice (FIG. 9A). Additional experiments were performed to examine whether signaling through the EP2 and EP4 receptors was altered in the absence of EP1. Following administration of PGE2, levels of cAMP levels were similarly enhanced in bone marrow cell cultures from wild type and EP1^{-/-} mice (FIG. 9B). Finally, immunohistochemistry was used to examine the expression of the EP4 receptor in fractures from wild type and EP1^{-/-} mice. Similar levels of expression were observed in the fractures from EP1^{-/-} and wild-type mice at 7, 14, and 21 days (FIG. 9C). Thus, the accelerated fracture healing observed in the absence of EP1 was independent of the EP2 and EP4 receptors.

### Example 5

### PGE2 Regulates Fibronectin Expression Specifically Through the EP1 Receptor

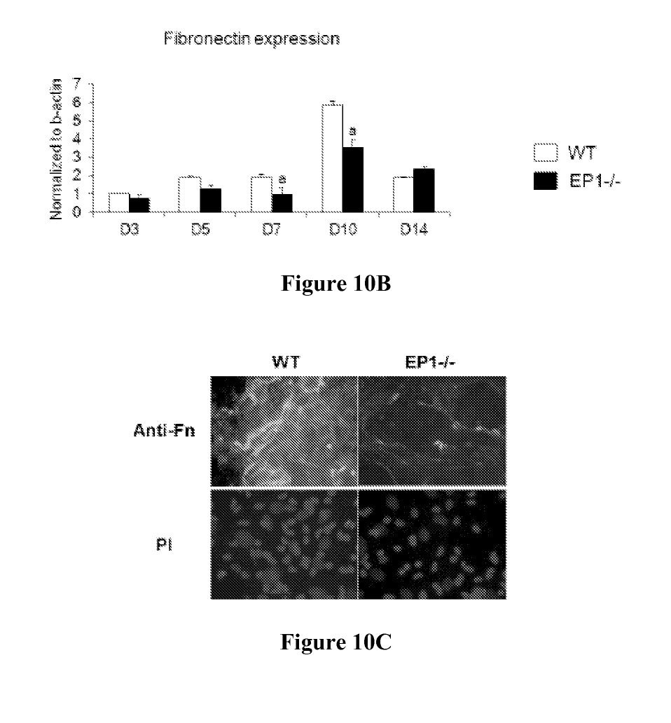

Fibronectin is an extracellular matrix protein shown to be essential for type I collagen polymerization. It was shown that in murine primary osteoblastic cells, Fibronectin is regulated by PGE2 specifically through the EP1 receptor. In order to confirm that this is the case in the bone marrow progenitor cell model described herein, Western blotting and real-time PCR were performed to examine the protein and mRNA levels of fibronectin, respectively. Basal levels of fibronectin were similar in wild-type and EP1^{-/-} cells. Upon treatment with PGE2, however, the expression of fibronectin is dramatically increased in wild-type cells compared with EP1^{-/-} cells (FIGS. 10A and 10B). Immunostaining was performed and showed that, with PGE2 treatment, wild-type cells expressed more fibronectin than EP1^{-/-} cells (FIG. 10C).

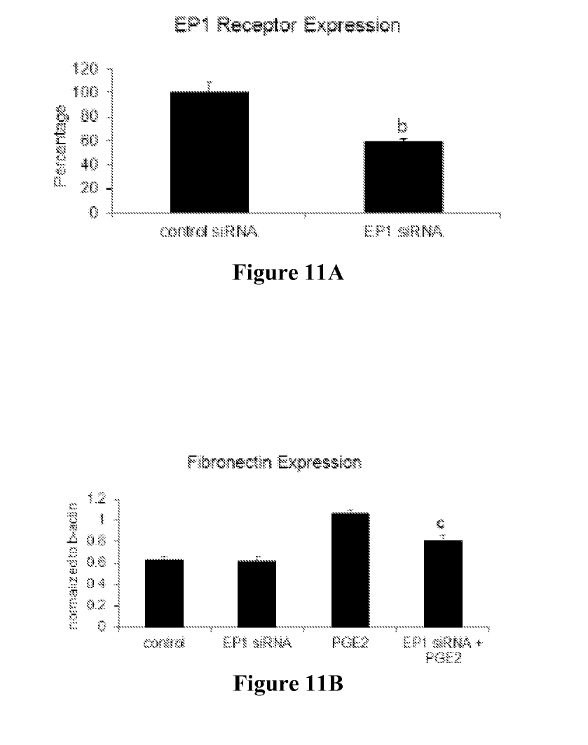

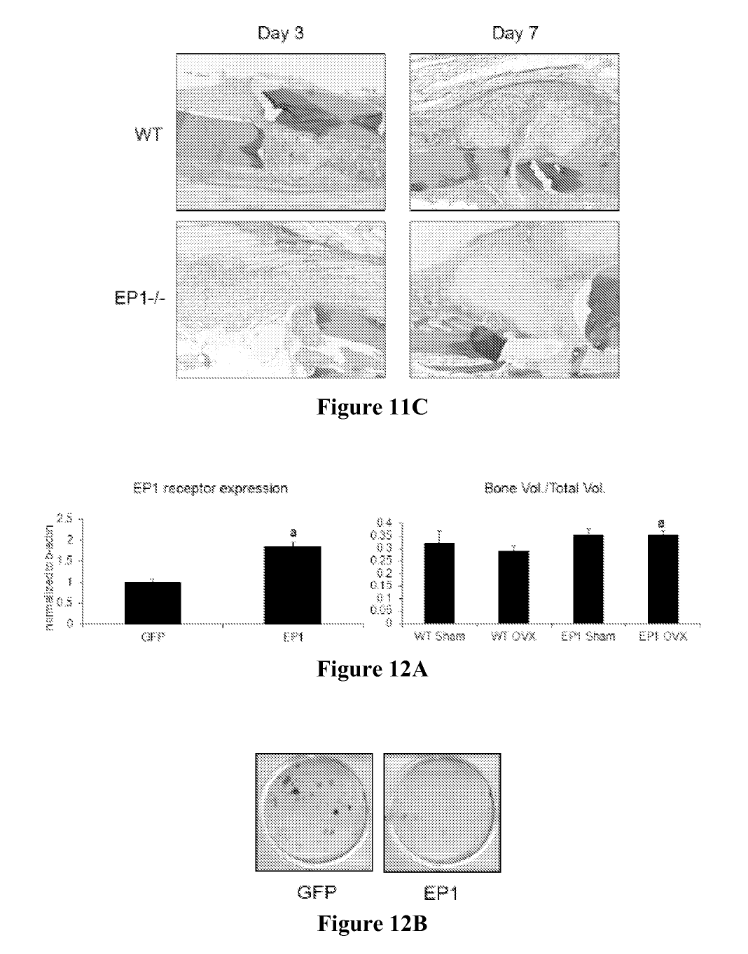

To further confirm regulation of fibronectin by the PGE2-EP1 signaling pathway, siRNA as described above was used to knock down EP1 expression in primary bone marrow stromal cells. Control siRNA conjugated with FITC was successfully transfected into these cells. EP1 mRNA was decreased about 40% by EP1 siRNA (FIG. 11A). Fibronectin expression levels were examined by quantitative real-time PCR. While PGE2 could stimulate fibronectin expression in cells with control siRNA, this expression was inhibited in cells transfected with EP1 siRNA (FIG. 11B).

Additionally, in vivo expression of fibronectin was assessed in the fracture callus from EP1^{-/-} and wild-type mice at days 3 and 7 post-fracture (FIG. 11C). Expression of fibronectin was observed in wild-type callus during the early fracture healing stage. According to cell morphology, Fibronectin is accumulated in the fibroblasts and non-hypertropic chondrocytes surrounding areas. However, the signal of fibronectin expression was completely absent in EP1^{-/-} fractures. This result further confirms that fibronectin expression is regulated by the PGE2-EP1 signaling pathway.

### Example 6

### EP1 is a Negative Regulator of Bone Formation

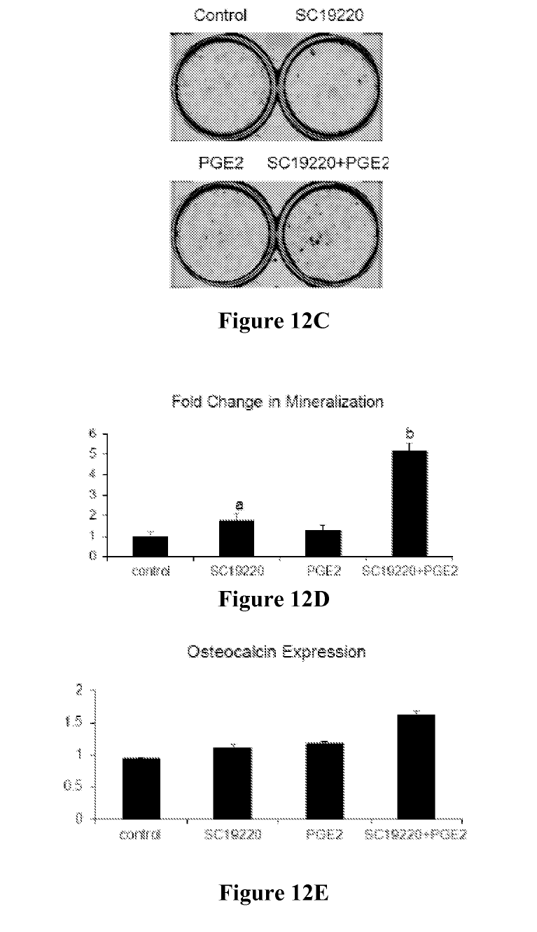

Since the in vivo studies showed that EP1^{-/-} mice have an increased bone formation rate and are resistant to bone loss, it was sought to determine whether over-expression of EP1 in EP1^{-/-} bone marrow stromal cells could rescue the knockout phenotype. An EP1 expression vector was co-transfected with an EGFP expression vector into primary bone marrow stromal cells. A transfection efficiency of 70% was observed. Both EP1 receptor and fibronectin expression were increased in the EP1^{-/-} cells (FIG. 12A). Alkaline phosphatase (ALP) staining at day 14 after transfection showed decreased ALP activity in the EP1 transfected cells, supporting that EP1 receptor expression negatively regulates osteoblast differentiation (FIG. 12B).

In contrast, treatment of primary bone marrow cell cultures with the EP1 antagonist SC19220 (Caymen Chemical, Ann Arbor, Mich.) accelerated bone nodule formation (FIGS. 12C and 12D). Wild-type primary bone marrow stromal cells in culture typically form bone nodules following 14 days in cell culture. However, treatment of 12 day cultures with SD19220 and PGE2 for 24 hours induced bone nodule formation by 13 days in culture (FIGS. 12C and 12D). The expression level of osteocalcin was increased in cultures treated to with SC19220 and PGE2 (FIG. 12E)

### Example 7

### Excessive Fibronectin Accumulation Impairs Osteoblast Differentiation

Since fibronectin is downstream of the PGE2-EP1 signaling pathway and it was previously shown that EP1 negatively regulates bone formation by inhibiting osteoblast differentiation, it is important to know whether fibronectin also has a negative effect on osteoblastogenesis. EP1^{-/-} bone marrow cells express a basal level of fibronectin similar to wild-type cells (FIGS. 10A and 10B). With PGE2 treatment, however, the induction of fibronectin expression is significantly lower in EP1^{-/-} cells compared to wild-type cells. Therefore, it was hypothesized that very high expression of fibronectin may inhibit osteogenesis.

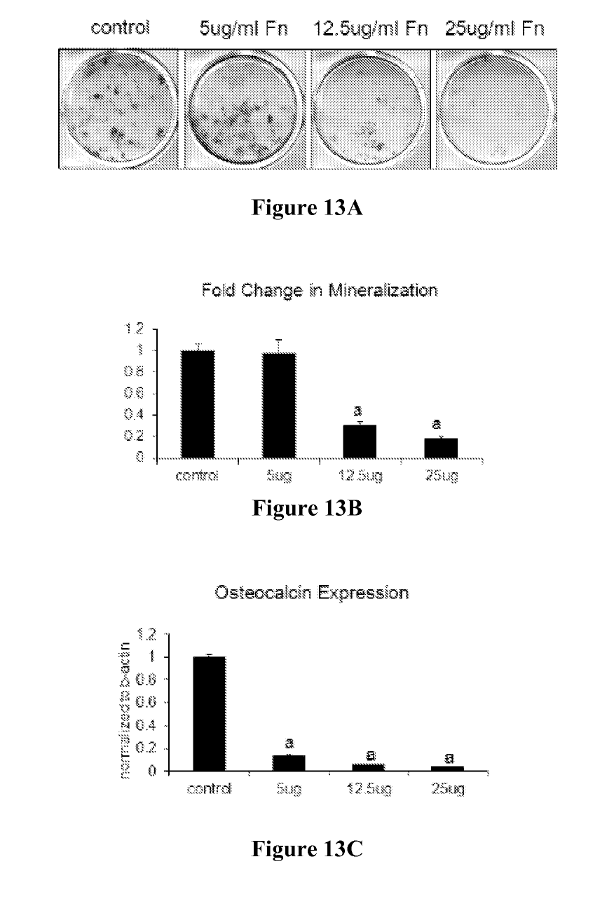

In order to assess the effect of fibronectin on osteoblast differentiation, wild-type bone marrow cells were treated with fibronectin at increasing concentrations. Addition of 5 μg/ml fibronectin did not alter bone nodule formation (FIGS. 13A and 13B). However, 12.5 μg/ml and 25 μg/ml fibronectin significantly reduced mineralization in a dose-dependent manner as assessed by alizarin red staining (FIGS. 13A and 13B), suggesting that excessive fibronectin inhibits osteoblast differentiation.

### Example 8

### EP1^{-/-} Mice have Increased Bone Mineral Density in Both Cortical and Trabecular Bone and Altered Bone Biomechanical Properties

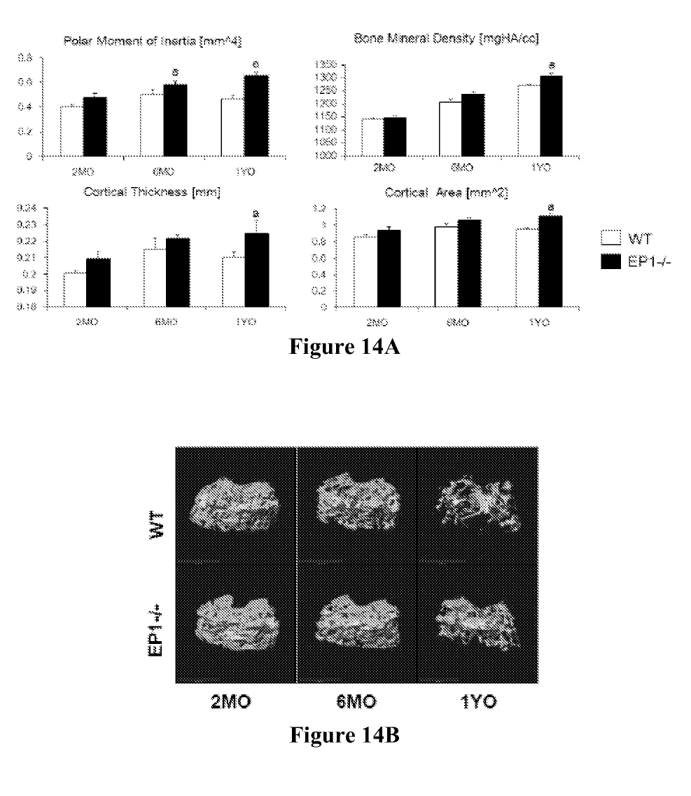

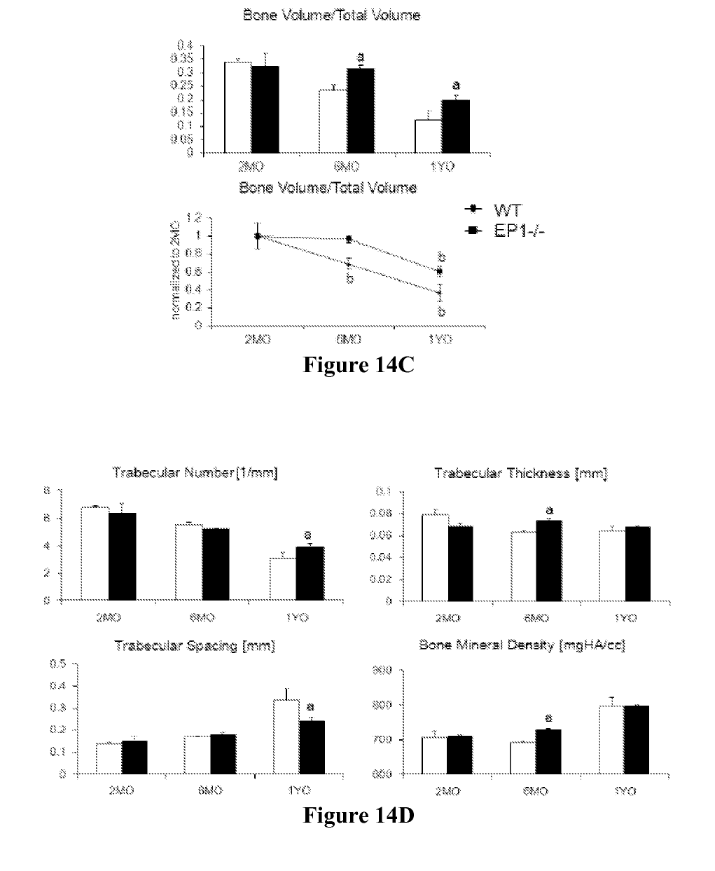

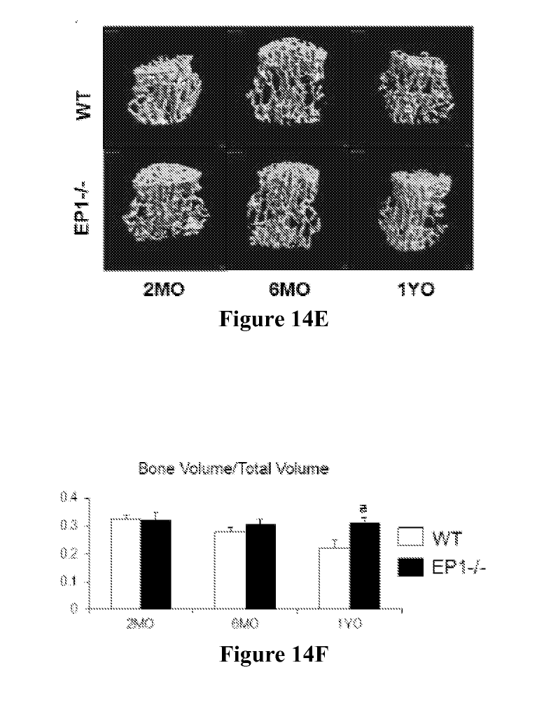

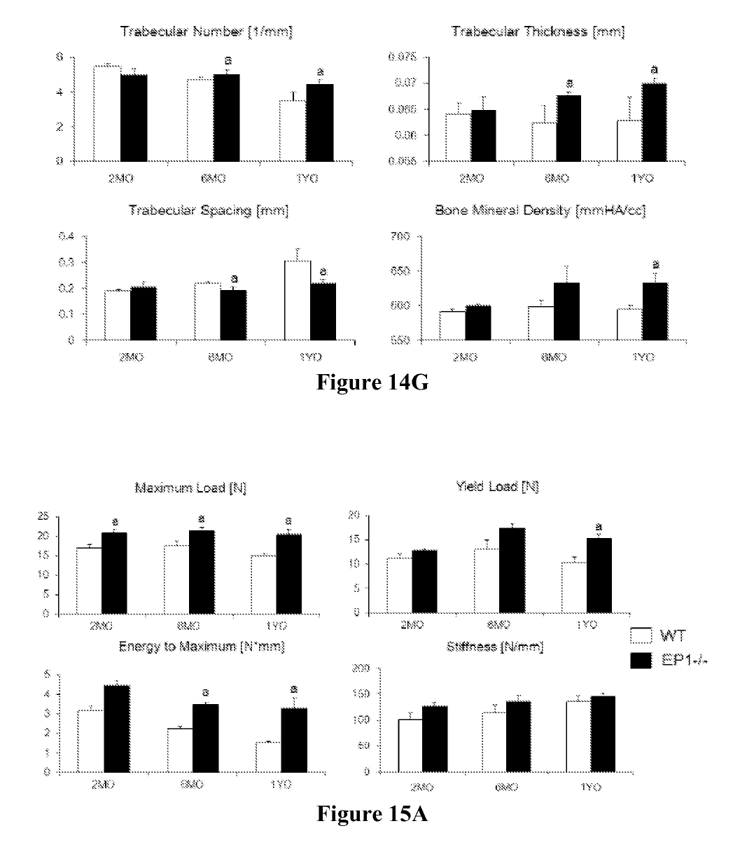

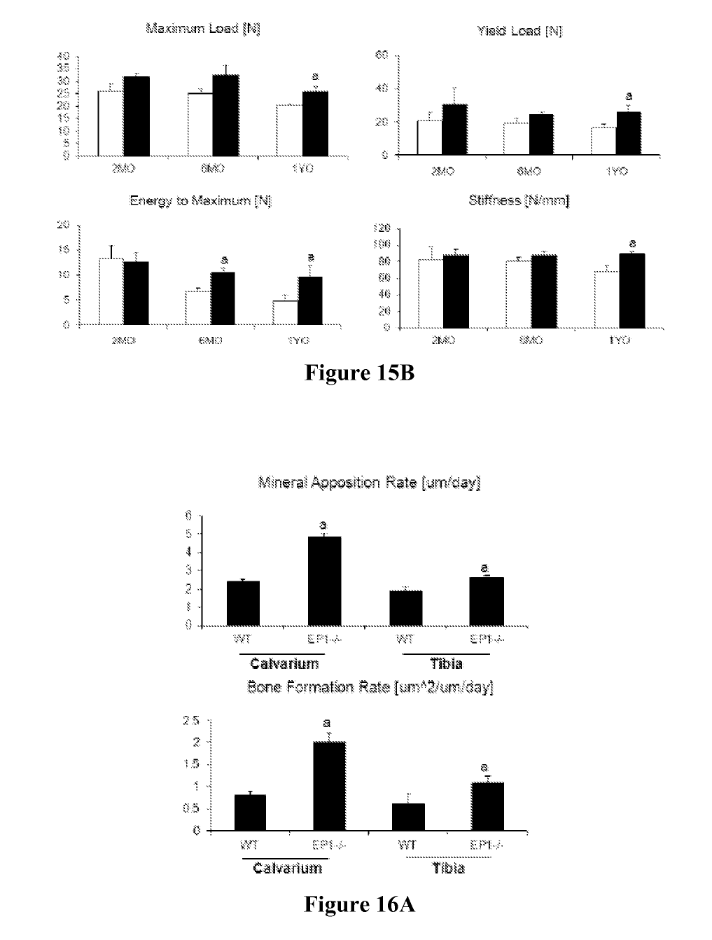

EP1^{-/-} mice exhibit accelerated fracture healing when compared to wild-type mice. Furthermore, bone mesenchymal progenitor cells from EP1^{-/-} mice form bone nodules faster than those from wild-type mice when cultured in mineralizing media. Therefore, it is likely that EP1^{-/-} mice have different bone properties compared to wild-type mice. Analysis of μ-CT data from the mid-diaphyseal region showed a significant increase of polar moment of inertia, bone mineral density, cortical thickness and cortical area in 1-year-old EP1^{-/-} mice compared to wild-type age-match control mice (FIG. 14A), indicating that EP1 has a significant impact on the cortical bone geometry. Consistently, analysis of the 3-point bending mechanical test results showed that 1-year-old EP1^{-/-} mice had significantly increased maximum load, yield load and energy to maximum (FIG. 15A).

Reconstruction of μ-CT data collected from the distal metaphyseal region of the femur showed that EP1^{-/-} mice had higher trabecular bone volume (FIGS. 14B and 14C). As age increases, wild-type mice gradually lose trabecular bone. However, in EP1^{-/-} mice, bone loss was delayed. 6-month-old EP1^{-/-} mice have a similar trabecular bone volume as 2-month-old EP1^{-/-} mice. Furthermore, 1-year-old EP1^{-/-} mice had significantly higher bone volume compared to 1-year-old wild-type mice. Besides total bone volume, EP1^{-/-} mice also showed increased trabecular number, trabecular thickness and bone mineral density and decreased trabecular spacing (FIG. 14D). Consistent with these findings, the trabecular region of the L-4 vertebrae of the EP1^{-/-} mice showed higher bone volume, trabecular number, trabecular thickness and bone mineral density as well as lower trabecular spacing (FIGS. 14E-14G). Compression test on the same L-4 vertebrae showed EP1^{-/-} mice had higher maximum load, yield load, energy to maximum and stiffness compared to wild-type mice of the same age (FIG. 15B).

### Example 9

### EP1^{-/-} Mice Show Increased Bone Formation and Normal Resorption

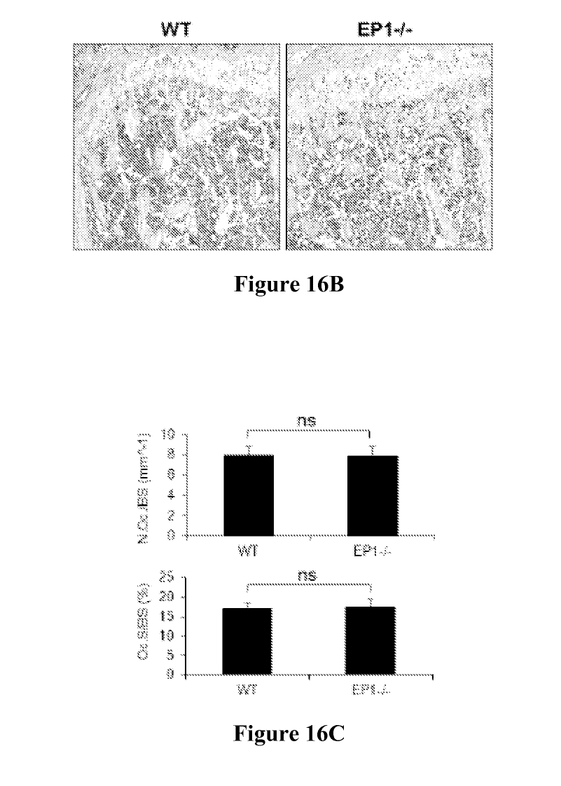

In order to determine whether the increased bone volume in the EP1^{-/-} mice is due to an increase in bone formation or a decrease in bone resorption, calcein labeling was performed to examine the bone formation rate. Calcein labeling showed significantly higher mineral apposition and bone formation rates in the 2-month-old EP1^{-/-} mice compared to wild-type mice (FIG. 16A).

To determine whether there was also a change in the bone resorption rate of the EP1^{-/-} mice, TRAcP staining was performed on sections from the metaphyseal bone adjacent to the growth plate. TRAcP staining showed a similar number of osteoclasts in the growth plate region of wild-type and EP1^{-/-} mice (FIG. 16B). Histomorphometric analysis of TRAcP stained sections showed similar osteoclast numbers and resorption surface between EP1^{-/-} and wild-type mice (FIG. 16C). Collectively, these results suggest that the change in bone properties of the EP1^{-/-} mice, especially the increased bone mineral density, is due to an increase in bone formation.

### Example 10

### EP1^{-/-} Mice are Resistant to OVX Induced Bone Loss

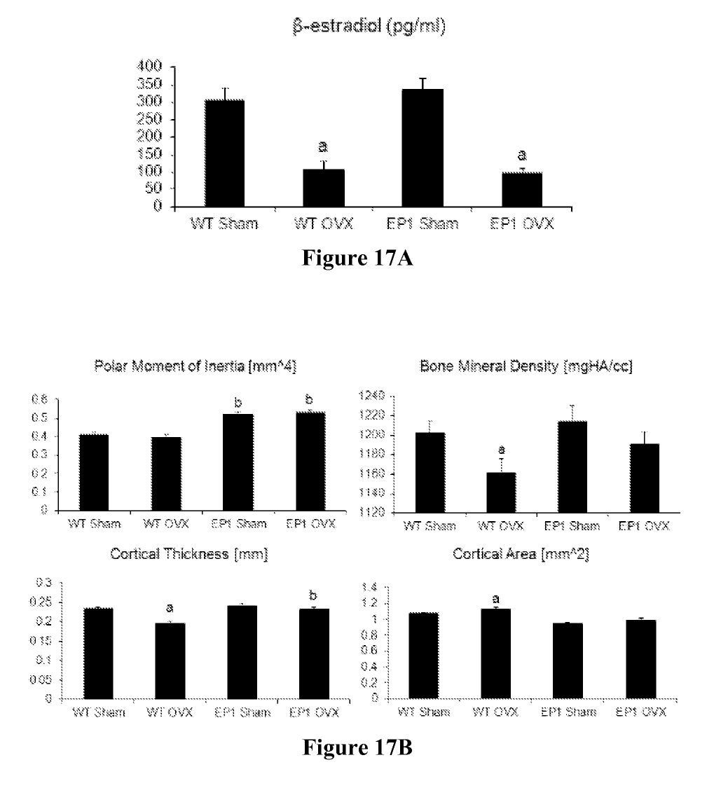

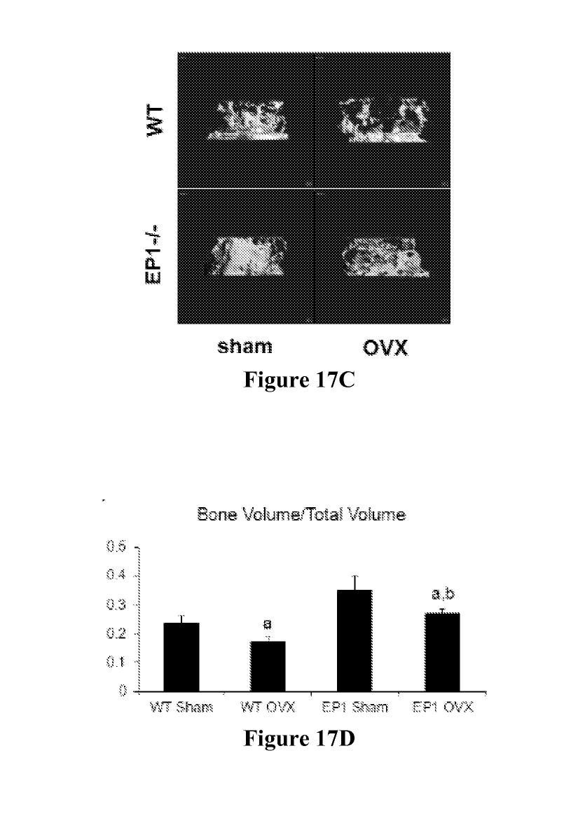

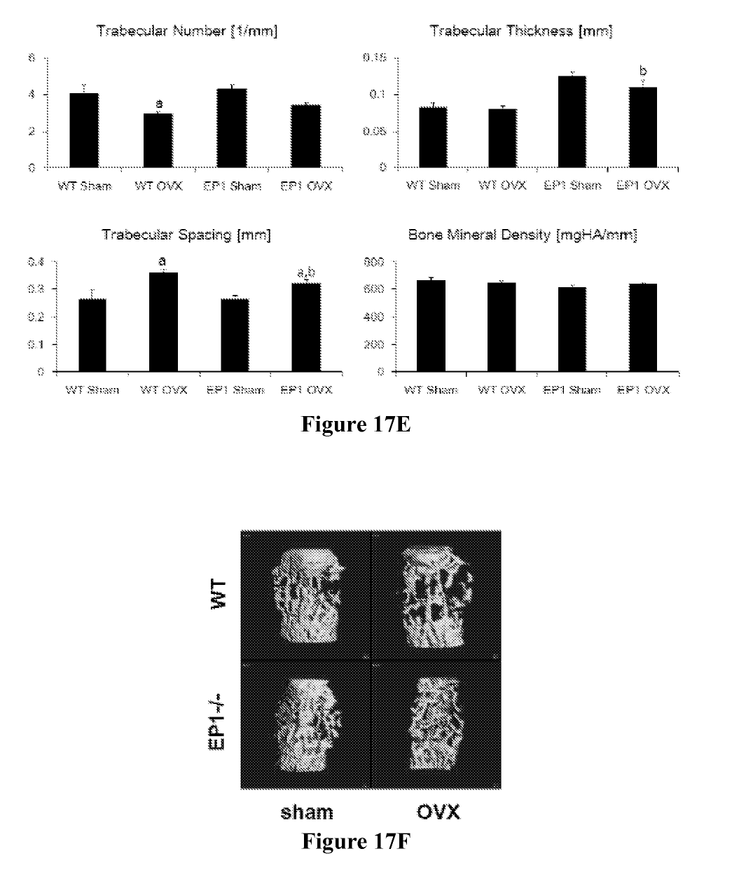

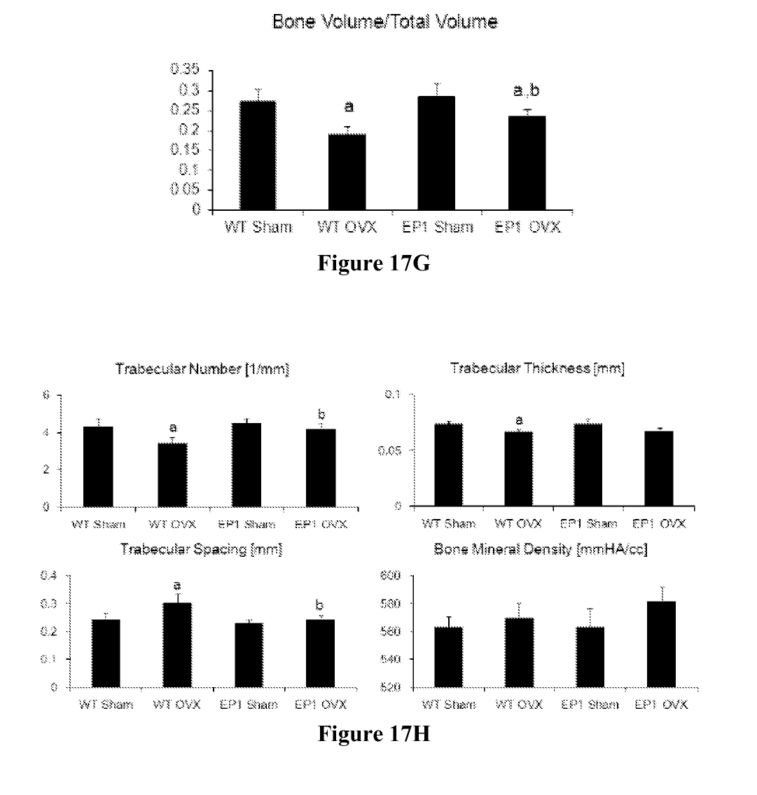

Osteoporosis is a condition characterized by progressive loss of bone density. Since EP mice show resistance to trabecular bone loss during aging, whether EP1^{-/-} mice may be also resistant to ovariectomy (OVX)-induced bone loss was further investigated. Following surgery, serum β-estradiol levels of sham control group mice and ovariectomized mice were determined. Both wild-type and EP1^{-/-} OVX mice showed a significant decrease in β-estradiol levels compared to mice who underwent sham surgery (FIG. 17A).

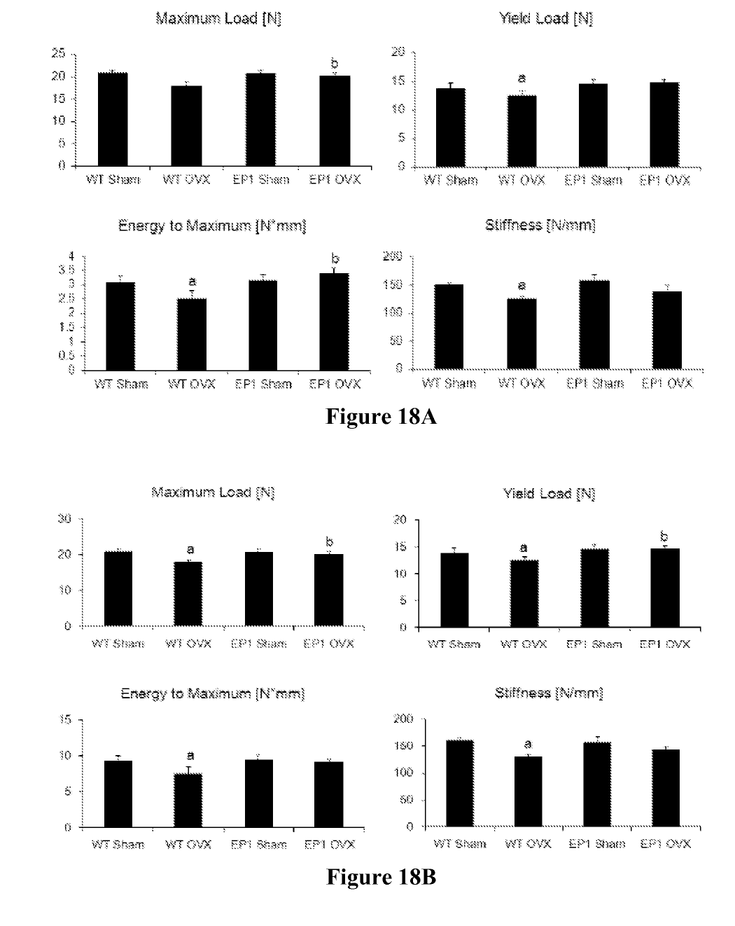

Micro-CT analysis and 3-point bending mechanical tests were performed to compare the cortical bone properties between EP1^{-/-} and wild-type mice with sham surgery or ovariectomy. Femurs from ovariectomized wild-type mice had less cortical thickness and bone mineral density than wild-type sham mice (FIG. 17B), resulting in a decrease of cortical bone biomechanical properties such as maximum load, yield load, energy to maximum and stiffness (FIG. 18A). Ovariectomized EP1^{-/-} mice, however, have similar bone properties compared to EP sham mice. Femurs from EP1^{-/-} OVX mice had significantly higher polar moment of inertia and cortical thickness, resulting to higher maximum load, and energy to maximum than those from wild-type OVX mice (FIG. 18A).

The metaphyseal regions of the femora and L-4 vertebrae were examined by μ-CT in order to analyze the trabecular bone properties (FIGS. 17B-17G). EP1^{-/-} and wild-type sham mice have similar bone volume and other bone parameters at two months following surgery (FIGS. 16B-16G). Wild-type OVX mice have significantly lower bone volume in the metaphysis of femur compared to wild-type sham mice confirming that ovariectomy accelerates trabecular bone loss. EP1^{-/-} OVX mice, however, have much more trabecular bone than wild-type OVX mice suggesting that EP1^{-/-} mice are resistant to the bone loss caused by ovariectomy. Moreover, compression tests performed on the L-4 vertebrae revealed that EP1^{-/-} mice are resistant to the change in maximum load, yield load, energy to maximum and stiffness caused by ovariectomy (FIG. 18B).

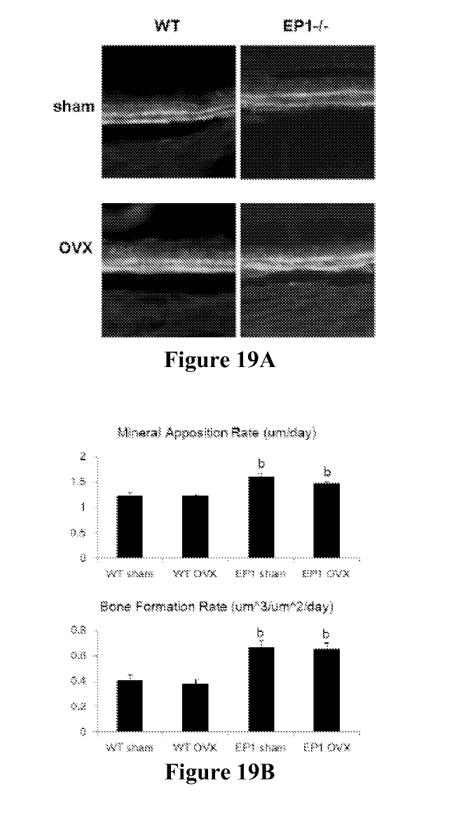

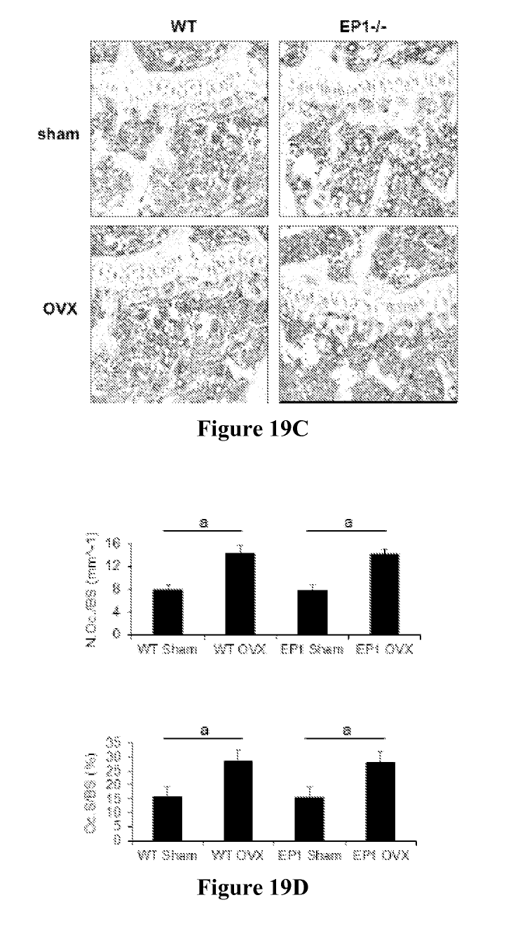

Osteoporosis is caused by the imbalance of bone formation and bone resorption. Therefore, the bone formation and resorption rates of the ovariectomized mice was examined. A significant decrease in bone formation in OVX mice compared to sham mice was shown by calcein labeling in both wild-type and EP1^{-/-} mice (FIG. 19A). Yet, the EP1^{-/-} OVX mice still have higher bone formation rate compared to wild-type OVX mice. On the other hand, TRAcP staining revealed a similar number of osteoclasts near the growth plate region of the OVX mice compared to sham mice by, showing no significant change in the resorption rate caused by ovariectomy (FIGS. 19B and 19C). Similarly, no difference in bone resorption was observed between EP1^{-/-} and wild-type mice subjected to either sham or ovariectomy surgery. These results indicate that EP1^{-/-} mice have a higher bone formation rate that compensates for the trabecular bone loss caused by ovariectomy. Furthermore, inhibition of EP1 receptor function protects against the osteopenia and bone loss that result from estrogen deficiency.

1. A method of accelerating bone fracture healing in a subject, the method comprising:
(a) identifying a subject with a bone fracture;
(b) administering to the subject an agent that inhibits a level of expression or activity of an EP1 receptor in an effective amount to accelerate the bone fracture healing in the subject.

2. (canceled)

3. The method of claim 1, wherein the agent is selected from the group consisting of a small molecule, a polypeptide, an antibody, a nucleic acid molecule, a peptidomimetic, or a combination thereof.

4. (canceled)

5. The method of claim 3, wherein the agent is a nucleic acid molecule selected from the group consisting of a short interfering RNA (siRNA) molecule, a microRNA (miRNA) molecule, or an antisense molecule.

6. (canceled)

7. The method of claim 5, wherein the nucleic acid molecule is an siRNA molecule comprising 5′-AGCUUGUCGGUAUCAUGGUTT-3′ (SEQ ID NO:21) or 5′-ACUUCUAAGCACACCAGATT-3′ (SEQ ID NO:22).

8-11. (canceled)

12. The method of claim 1, further comprising administering to the subject a second agent that regulates osteoblast or osteoclast differentiation or function.

13. The method of claim 12, wherein the regulation of osteoblast or osteoclast differentiation or function is determined by the level of one or more markers of osteoblast or osteoclast differentiation or function.

14. The method of claim 13, wherein the one or more markers of osteoblast differentiation or function is selected from the group consisting of alkaline phosphatase, runx2, osterix, osteocalcin, bone sialoprotein, and type 1 collagen.

15. The method of claim 13, wherein the one or more markers of osteoclast differentiation or function is selected from the group consisting of tartrate resistant acid phosphotase (TRAP), cathepsin K, and calcitonin receptor.

16. (canceled)

17. A method of screening for an agent that accelerates bone fracture healing or treats or prevents osteoporosis, the method comprising:
(a) providing a cell of osteocyte lineage comprising an EP1 receptor;
(b) contacting the cell with an agent to be screened; and
(c) determining a level of expression or activity of the EP1 receptor in the cell, wherein a decrease in the level of expression or activity of the EP1 receptor as compared to a control indicates the agent accelerates bone fracture healing or treats or prevents osteoporosis.

18. The method of claim 17, wherein the agent is selected from the group consisting of a small molecule, a polypeptide, an antibody, a nucleic acid molecule, a peptidomimetic, or a combination thereof.

19-27. (canceled)

28. A method of screening for an agent that accelerates bone fracture healing in a subject, the method comprising:
(a) administering to the subject an agent to be screened, wherein the subject has a bone fracture; and
(b) determining whether the agent inhibits the level of expression or activity of an EP1 receptor at the site of the bone fracture, a decrease in the level of expression or activity of the EP1 receptor as compared to a control indicates the agent accelerates bone fracture healing.

29. The method of claim 28, wherein the agent is selected from the group consisting of a small molecule, a polypeptide, an antibody, a nucleic acid molecule, a peptidomimetic, or a combination thereof.

30-42. (canceled)

43. A method of treating or preventing osteoporosis in a subject, the method comprising:
(a) identifying a subject with or at risk of developing osteoporosis;
(b) administering to the subject an agent that inhibits the level of expression or activity of an EP1 receptor in an amount effective to treat osteoporosis.

44. (canceled)

45. The method of claim 43, wherein the agent is selected from the group consisting of a small molecule, a polypeptide, an antibody, a nucleic acid molecule, a peptidomimetic, or a combination thereof.

46. (canceled)

47. The method of claim 45, wherein the agent is a nucleic acid molecule selected from the group consisting of a short interfering RNA (siRNA) molecule, a microRNA (miRNA) molecule, or an antisense molecule.

48. (canceled)

49. The method of claim 47, wherein the nucleic acid molecule is an siRNA molecule comprising 5′-AGCUUGUCGGUAUCAUGGUTT-3′ (SEQ ID NO:21) or 5′-ACUUCUAAGCACACCAGATT-3′ (SEQ ID NO:22).

50-53. (canceled)

54. The method of claim 43, further comprising administering to the subject a second agent that regulates osteoblast or osteoclast differentiation or function.

55. The method of claim 54, wherein the regulation of osteoblast or osteoclast differentiation or function is determined by the level of one or more markers of osteoblast or osteoclast differentiation or function.

56. The method of claim 54, wherein the one or more markers of osteoblast differentiation or function is selected from the group consisting of alkaline phosphatase, runx2, osterix, osteocalcin, bone sialoprotein, and type 1 collagen.

57. The method of claim 54, wherein the one or more markers of osteoclast differentiation or function is selected from the group consisting of tartrate resistant acid phosphatase (TRAP), cathepsin K, and calcitonin receptor.

58. The method of claim 56, wherein the marker of osteoblast differentiation or function is alkaline phosphatase.

59. A method of screening for an agent that treats or prevents osteoporosis in a subject, the method comprising:
(a) administering to the subject an agent to be screened, wherein the subject has or is at risk of developing osteoporosis; and
(b) determining whether the agent inhibits the level of expression or activity of an EP1 receptor, a decrease in the level of expression or activity of the EP1 receptor as compared to a control indicates the agent treats or prevents osteoporosis.

60. The method of claim 59, wherein the agent is selected from the group consisting of a small molecule, a polypeptide, an antibody, a nucleic acid molecule, a peptidomimetic, or a combination thereof.

61-70. (canceled)


SEQUENCE LISTING (26 sequences)

SEQ ID NO: 1 - DNA, 20 bp, Artificial Sequence
  Synthetic Construct
agatgtggatcagcaagcag

SEQ ID NO: 2 - DNA, 20 bp, Artificial Sequence
  Synthetic Construct
gcgcaagttaggttttgtca

SEQ ID NO: 3 - DNA, 18 bp, Artificial Sequence
  Synthetic Construct
gccaaggcaacagtcgct

SEQ ID NO: 4 - DNA, 24 bp, Artificial Sequence
  Synthetic Construct
cttggtggttttgtattcgatgac

SEQ ID NO: 5 - DNA, 20 bp, Artificial Sequence
  Synthetic Construct
actggtaagtggggcaagac

SEQ ID NO: 6 - DNA, 20 bp, Artificial Sequence
  Synthetic Construct
ccacaccaaattcctgttca

SEQ ID NO: 7 - DNA, 20 bp, Artificial Sequence
  Synthetic Construct
ctttgtgtgcctttcaatcg

SEQ ID NO: 8 - DNA, 23 bp, Artificial Sequence
  Synthetic Construct
gtgaggtacagcctaccagtttt

SEQ ID NO: 9 - DNA, 18 bp, Artificial Sequence
  Synthetic Construct
agggaggatcaagtcccg

SEQ ID NO: 10 - DNA, 18 bp, Artificial Sequence
  Synthetic Construct
gaacagactccggcgcta

SEQ ID NO: 11 - DNA, 20 bp, Artificial Sequence
  Synthetic Construct
tgaccttctctcctccatcc

SEQ ID NO: 12 - DNA, 20 bp, Artificial Sequence
  Synthetic Construct
cttcctgggagtctcatcct

SEQ ID NO: 13 - DNA, 20 bp, Artificial Sequence
  Synthetic Construct
gccgggaatgatgagaacta

SEQ ID NO: 14 - DNA, 20 bp, Artificial Sequence
  Synthetic Construct
ggaccgtccactgtcacttt

SEQ ID NO: 15 - DNA, 22 bp, Artificial Sequence
  Synthetic Construct
gtcaagagtcttagccaaactc

SEQ ID NO: 16 - DNA, 21 bp, Artificial Sequence
  Synthetic Construct
aaatgatgtgaggccagatgg

SEQ ID NO: 17 - DNA, 20 bp, Artificial Sequence
  Synthetic Construct
caccatcagctgaagatagt

SEQ ID NO: 18 - DNA, 20 bp, Artificial Sequence
  Synthetic Construct
ccaagatctctaacatgacg

SEQ ID NO: 19 - DNA, 19 bp, Artificial Sequence
  Synthetic Construct
agtccgtgaagcaggagtg

SEQ ID NO: 20 - DNA, 22 bp, Artificial Sequence
  Synthetic Construct
ccatctggacattttttgcaaa

SEQ ID NO: 21 - DNA, 21 bp, Artificial Sequence
  Synthetic Construct
agcuugucgguaucauggutt

SEQ ID NO: 22 - DNA, 20 bp, Artificial Sequence
  Synthetic Construct
acuucuaagcacaccagatt

SEQ ID NO: 23 - DNA, 20 bp, Artificial Sequence
  Synthetic Construct
tacatgggatgctcgaaaca

SEQ ID NO: 24 - DNA, 20 bp, Artificial Sequence
  Synthetic Construct
ttttaagcccgtgtgggtag

SEQ ID NO: 25 - DNA, 21 bp, Artificial Sequence
  Synthetic Construct
gtgaagaacgaggaggatgtg

SEQ ID NO: 26 - DNA, 20 bp, Artificial Sequence
  Synthetic Construct
gtgatggcggatgatgtagc